(19) United States
(12) Patent Application Publication
(10) Pub. No.: US 2026/0363696 A1
(43) Pub. Date: Nov. 19, 2026

(54) SUTURING DEVICES AND METHODS FOR TISSUE CLOSURE

(71) Applicants: Luis Bogran, Kenner, LA (US); Jon Bogran, Kenner, LA (US)

(72) Inventors: Luis Bogran, Kenner, LA (US); Jon Bogran, Kenner, LA (US)

(21) Appl. No.: 19/749,543

(22) Filed: Jul. 21, 2026

Related U.S. Application Data
(63) Continuation-in-part of application No. 17/834,816, filed on Jun. 7, 2022, now Pat. No. 12,721,621.
(60) Provisional application No. 63/326,216, filed on Mar. 31, 2022.

(51) Int. Cl. A61B 17/04; A61B 17/00

(52) U.S. Cl. CPC A61B 17/0469; A61B 2017/00876; A61B 2017/047

(57) ABSTRACT

A suturing device and methods are disclosed herein. The suturing device may include an elongated body having proximal and distal ends and defining a longitudinal axis. A curved needle assembly may be arranged at the distal end and may be movable between at least an extended position and a retracted position. The curved needle assembly may be configured, in the extended position, to advance a first portion of a suture along a suture path through tissue, and may be configured, in the retracted position, to retract the first portion of the suture along the suture path. The suturing device may further include a control assembly arranged at the proximal end of the elongated body and may be configured to move the curved needle assembly.

### CROSS-REFERENCES TO RELATED APPLICATIONS

This application is a continuation-in-part application and claims benefit of priority of U.S. patent application Ser. No. 17/834,816, filed Jun. 7, 2022, which claims the benefit of priority of U.S. Provisional Patent Application No. 63/326,216, titled “HANDHELD MEDICAL SUTURING DEVICE” and filed Mar. 31, 2022, the entirety of each application is incorporated herein by reference.

### BACKGROUND

### Field of the Invention

The present invention generally relates to a suturing instrument for joining two edges of biological tissue together and more particularly relates to a handheld medical suturing device for joining two edges of biological tissue together with a curved needle and tying knots or locking sutures.

### Description of the Related Art

People around the world encounter lacerations and other broken tissues in need of medical suturing (stitching). During operations, surgeons make a cut (incision) in the skin to gain access to the surgical site, or an incision already exists due to an injury. Incisions are closed with sutures (stitches), staples, tissue adhesives or tapes. One of the problems inherent in many surgical procedures is that of limited access of the surgeon's hands, as well as the needle and suturing implements, into the incision or wound. In performing surgical procedures, surgeons often rely on existing methods of manually suturing and tying knots that are slow, time consuming and tedious. In conventional methods, a patient's wounds can be left open longer than desired, which increases the risk of infection or other complications. It is well known that impaired wound healing increases costs of health care and leads to poor cosmetic results (Continuous versus interrupted skin sutures for nonobstetric surgery, Gurusamy K S, Toon C D, Allen V B, Davidson B R, Cochrane Database Syst Rev. 2014 Feb 14). Also, existing methods and devices further can cause fatigue and repetitive physical strain ailments or injuries for surgeons when manually manipulating existing tools to suture wounds and tie knots.

These problems are amplified under circumstances where delicate surgery such as heart, brain, and spinal surgery, as well as surgery on infants and children is undertaken, since the surgical areas of interest involve minute features. Such conventional methods include the use of tools such as small curved needles, needle drivers, tweezers, and clamps to secure sutures and close external wounds. Many surgical procedures that would otherwise be possible on adults and children are impossible due to the tiny operating fields, and many conditions that might otherwise be corrected by surgery are, therefore, considered to be inoperable. The same situation occurs under circumstances such as suturing within interior and normally inaccessible areas of the body where no known surgical techniques and/or instruments can access these areas and provide the necessary surgical relief

Moreover, incisional ventral hernias are the most common complication after a laparotomy (open abdomen surgery) and are the most common indication for reoperation after laparotomy, but roughly half of these hernias are preventable if better suturing techniques are implemented, including smaller sutures that are more closely placed together. More than 150,000 patients a year undergo incisional ventral hernia repair in the United States alone, at an approximate cost of one billion dollars. Studies of the effects of changes to our usual method of closing abdominal surgical wounds by using the techniques for continuous suturing of midline abdominal wound incisions proposed by Leif Israelsson et al. have shown that smaller sutures (6 mm to 8 mm from the fascial wound edge) that are more closely placed together to yield a suture-to-wound length ratio of 4:1 or greater cut the incidence of incisional hernias by half This has been verified by subsequent studies and indicates that about half the incisional hernias are preventable when surgeons implement smaller sutures that are more closely placed together and would save much patient suffering and as much as half a billion dollars a year in surgical expenses. Thus, a need exists for new medical devices and methods for placing sutures more closely together.

Various devices and methods have been discovered over the years for suturing and other surgical techniques, such as those disclosed in U.S. Pat. No. 4,417,532 to Yasukata, U.S. Pat. No. 4,557,265 to Anderson, U.S. Pat. No. 4,899,746 to Brunk, U.S. Pat. No. 5,364,408 to Gordon, U.S. Pat. No. 5,458,609 to Gordon et al, U.S. Pat. No. 5,540,704 to Gordon et al., U.S. Pat. No. 5,578,044 to Gordon et al., U.S. Pat. No. 6,641,592 to Sauer et al., U.S. Patent Publication No. 2010/0042116 to Chui et al., U.S. Pat. No. 8,628,545 to Cabrera et al., U.S. Pat. No. 9,386,980 to Smith, and U.S. Pat. No. 9,554,793 to Lane et al. While these units may be suitable for the particular purpose employed, they would not be as suitable, efficient, and safe for the purposes and achievements of the present invention as disclosed hereafter.

Consequently, one object of the present invention is to provide a suturing apparatus that can be used readily, even in cases of laparoscopy surgery, and which sews a suture by threading a suture material through a patient's tissue and also ties a knot or locks the suture at intervals when suturing the tissues together, both with respect to a single surgical stitch and a row of such stitches.

It therefore can be seen that a need exists for new surgical devices and methods that address the foregoing and other related and unrelated problems in the art.

A need exists for medical suturing devices that will help insert the suturing materials through tissues while alleviating or obviating the fatigue otherwise associated with manually suturing and tying knots. A need exists for new and improved medical suturing devices of comparatively simple construction and arrangement, and where the medical suturing devices are strong, durable, and efficient in its use.

As disclosed in this application, the inventor has discovered novel and unique medical devices and methods for suturing tissues and fabrics, which exhibit superlative properties without being dependent on manual, immobile, components.

The devices and methods disclosed herein avoid many of the drawbacks of existing methods and devices which rely on expensive complexities or complex tools for application of the suturing materials to tissue or fabrics.

Embodiments of the present invention provide for improved medical suturing devices and methods as described and defined in the description below and in the annexed claims which provide for improved suturing and knot tying operation characteristics with reduced fatigue in order to efficiently suture a patient's tissue or intended fabrics. Each patent application identified above is incorporated here by reference in its entirety.

### SUMMARY OF THE INVENTION

It is one prospect of the present invention to provide novel medical suturing devices and methods of simple but effective construction and operation which can be applied to varying sizes of tissue lacerations and other openings to securely suture or bind tissues in an efficient and effective manner.

Further objects of the invention are to provide medical suturing devices and processes which are comparatively simple in its construction and arrangement, strong, durable, efficient in its use, readily set up in operative position with respect to tissue or fabrics, and comparatively inexpensive to manufacture. Another object is to provide for medical suturing devices that are single use. It is also an aim of the present invention to provide an improved suturing medical suturing system that provides improved ergonomic use and is of economical construction while providing nearly universal suturing operations without surgical fatigue, overcoming the complexities and limitations of conventional suturing devices and procedures.

Another object of the present invention is to provide a medical suturing device which provides knot tying of suturing material in line with the action of insertion of suturing material through a patient's tissue.

Additional objects, advantages and novel features of this invention shall be set forth in the description that follows, and in part will become apparent to those skilled in the art upon examination of the following specification. The objects and advantages of the invention may be realized and attained by means of the instrumentalities, combinations, and devices particularly pointed out in the appended claims.

The following presents a simplified summary of the present disclosure in a simplified form as a prelude to the more detailed description that is presented herein.

Therefore, to achieve the foregoing and other objects and in accordance with the purposes and embodiments of the present invention, as embodied and described herein, there is provided a handheld medical suturing device comprising a handle body having a hinge operatively connected to a distal end of a handle lever. The handle lever is adapted to be grasped by a hand of a user. The handle body has proximal end adapted to be grasped by a user and a distal end which defines an opening adapted to receive a tissue.

In an embodiment, the handheld medical suturing device includes an actuating arm disposed within the handle body. The actuating arm has a proximal end and a distal end, and the proximal end of the actuating arm is fixed to the distal end of the handle lever.

In one embodiment, the handheld medical suturing device includes a bent constrained rigid body, which has a proximal end and a distal end. The proximal end of the bent constrained rigid body is operatively connected to the actuating arm. The distal end of the bent constrained rigid body is operatively connected to a pivoting radial arm.

The pivoting radial arm has a proximal end opposite a distal end. The proximal end of the pivoting radial arm is operatively connected to a fulcrum point disposed within the handle body. The distal end of the pivoting radial arm is operatively connected to the distal end of the bent constrained rigid body.

In an embodiment, the handheld medical suturing device includes a curved suturing needle assembly disposed within the handle body. The curved suturing needle assembly has a proximal end and a distal end. The proximal end is operatively connected to the distal end of the pivoting radial arm. The curved suturing needle is adapted to advance a suture through the tissue by a rotation of the actuating arm and respective rotation of the bent constrained rigid body along an XY plane that is substantially perpendicular to the tissue. Such rotation rotates the curved suturing needle assembly through the tissue between a retracted position and a fully extended position upon a pivoting of the handle lever relative to the handle body from an open position to a squeezed position.

In one embodiment, the handheld medical suturing device further includes at least one spool of suture disposed within an inner cavity passageway defined by the proximal end of the handle body. Preferably, the handle body comprises an internal rectangular tube having walls that define the inner cavity passageway. The suture is threaded from the at least one spool through the inner cavity passageway of the handle body to the curved suturing needle assembly, and the suture is further threaded from the curved suturing needle assembly to a bobbin that is disposed within the distal end of the handle body of the handheld medical suturing device. The at least one spool is configured to dispense the suture to the curved suturing needle assembly upon the rotation of the curved suturing needle from the retracted position to the fully extended position.

In yet another embodiment, the handheld medical suturing device further includes a fixed bobbin channel that has a semi-annular body, which forms a bobbin channel cavity disposed within the distal end of the handle body. The semi-annular body defines an elongated slot therethrough, preferably located at the bottom of the semi-annular body of the fixed bobbin channel. The semi-annular body has a proximal end opposite a distal end, and the bobbin channel cavity has a central axis that is perpendicular to the XY plane.

In one embodiment, the handheld medical suturing device further comprises a bobbin carrier that is movably fitted within the bobbin channel cavity of the fixed bobbin channel. The bobbin carrier comprises a semi-annular body that forms a bobbin carrier cavity that is configured to receive the curved suturing needle assembly in the fully extended position. The bobbin carrier is adapted for movement of the bobbin along the central axis across the XY plane, between a rest position and an actuated position, as the bobbin is rotatably disposed within the bobbin carrier cavity of the bobbin carrier.

In an embodiment, the bobbin carrier of the handheld medical suturing device comprises a rack disposed along a bottom of the bobbin carrier. The rack is operatively fitted within the elongated slot of the fixed bobbin channel.

In yet another embodiment, the handheld medical suturing device further includes a mutilated spur gear that is fixed to a first drive axle disposed inside the handle body. In another embodiment, the handheld medical suturing device includes an unmutilated spur gear, rather than a mutilated spur gear.

Preferably, the first drive axle is connected to a first thumb lever that extends outwardly of the handle body. The mutilated spur gear operatively engages the rack to translate and impart rotational motion of the mutilated spur gear into linear motion of the rack from the rest position to the actuated position, upon the rotation of the first thumb lever from a first thumb lever first position to a first thumb lever second position. The linear motion of the rack moves the bobbin carrier side to side within the bobbin channel.

In an embodiment, the bobbin of the handheld medical suturing device has opposing hemispherical heads and a cylindrical body that forms an axle disposed between such opposing hemispherical heads. That axle is adapted to contain the suture between the opposing hemispherical heads. The cylindrical body is adapted to release the suture therefrom upon the movement of the bobbin carrier from the rest position to the actuated position, such that the bobbin is moved across the XY plane, preferably when the curved needle suturing assembly is in the fully extended position. The movement of the bobbin carrier across the XY plane moves the suture through a bow that is formed by the suture extending from the curved suturing assembly.

In one embodiment, the handheld medical suturing device includes at least one spring biasing member that is disposed within the inner cavity passageway of the handle body. The spring biasing member is preferably positioned between and connected to the at least one spool and an inner wall of the proximal end of the handle body. Such at least one biasing member tensions the suture between the at least one spool and the bobbin.

In another embodiment, the handheld medical suturing device further includes at least one spring biasing member that is disposed around the hinge of the handheld medical suturing device. Such at least one spring biasing member is operatively connected to the handle body and the distal end of the handle lever at the hinge, and such at least one biasing member pivotally biases the proximal end of the handle lever away from the proximal end of the handle body.

In an embodiment, the handheld medical suturing device further comprises a first spur gear that is concentrically disposed around and fixed to the first drive axle. The handheld medical suturing device further comprises a second spur gear concentrically disposed around and fixed to a second drive axle. The second drive axle is connected to a second thumb lever, which extends outward of the handle body. The first spur gear operatively engages the second spur gear. Similarly, the second spur gear engages the first spur gear to impart rotational movement.

In another embodiment, the handheld medical suturing device further comprises at least one locking pin fixed to the first thumb lever. Preferably, the handle lever defines a channel, wherein in the squeezed position and in the actuated position, the at least one locking pin aligns with the channel upon the rotation of the first thumb lever from the first thumb lever first position to the first thumb lever second position; an opening of the handle lever causes the locking pin to penetrate and slide into the channel of the handle lever, and such penetration of the locking pin in the channel of the handle lever obstructs a second pivoting of the handle relative to the handle body from the squeezed to the open position.

In such embodiment, the at least one locking pin of the handheld medical suturing device is configured for withdrawal from the channel upon a rotation of the first thumb lever by a user from the first thumb lever second position to the first thumb lever first position.

In an embodiment, the handheld medical suturing device further comprises at least one spool of the suture that is disposed within an inner cavity passageway that is defined by the proximal end of the handle body. The suture is preferably threaded from the at least one spool through the inner cavity passageway to the curved suturing needle assembly. The suture is further threaded from the curved suturing needle assembly to a bobbin that is disposed within the distal end of the handle body. The at least one spool is adapted to dispense the suture to the curved suturing needle assembly upon the rotation of the curved suturing needle by a user from the retracted position to the fully extended position.

In such embodiment, the bobbin has a proximal end opposite a distal end, and the proximal end preferably comprises a first plurality of annularly disposed ridges that are configured to provide a first frictional resistance against rotation of the bobbin within the bobbin carrier upon a rotation of the curved suturing needle assembly through the tissue, between the retracted position and the fully extended position, in order to keep the suture taut, upon the pivoting of the handle lever relative to the handle body. The frictional resistance of the first plurality of annularly disposed ridges also maintains tension on the suture upon the movement of the bobbin along the central axis across the XY plane between a rest position and an actuated position.

In another embodiment, the distal end of the bobbin comprises a second plurality of annually disposed ridges that provide a second frictional resistance against rotation of the bobbin relative to the bobbin carrier upon the rotation of the curved suturing needle assembly through the tissue, between the retracted position and the fully extended position, upon the pivoting of the handle lever relative to the handle body. Such second frictional resistance is also adapted to maintain tension on the suture upon a user's movement of the bobbin along the central axis across the XY plane between a rest position and an actuated position.

In yet another embodiment, the handheld medical suturing device has at least one magnet disposed within a wall of the bobbin carrier, in a spaced relation to the bobbin, and the bobbin comprises a ferrous material that is configured for a magnetic attraction, to provide a frictional resistance against rotation of the bobbin relative to the bobbin carrier, in order to keep the suture taut.

In another embodiment, the handheld medical suturing device preferably comprises at least one magnet that is disposed within a wall of the handle body, and the handheld medical suturing device further comprises at least one spool of suture that is disposed within an inner cavity passageway that defined by the proximal end of the handle body, and the at least one spool comprises a ferrous material that is adapted to provides a magnetic attraction to said at least one magnetic disc configured to provide a frictional resistance against rotation of the at least one spool relative to the wall of the handle body, in order to keep the suture taut.

In an embodiment, the curved suturing needle assembly of the handheld medical suturing device comprises an outer curved needle fixed to an inner curved needle. The outer curved needle has a proximal end opposite a distal end. The inner curved needle has a proximal end opposite a distal end. The curved suturing needle assembly preferably includes a shroud that is disposed along an outer surface of the outer curved needle and of the inner curved needle. The shroud forms a passage for the suture to thread therethrough.

The shroud includes a pointed cover encapsulating the distal end of the outer curved needle.

In yet another embodiment, the handheld medical suturing device includes an elbow guide disposed within the pointed cover. The elbow guide engages and guides the suture when the suture extends outwardly from the distal end of the inner curved needle. The distal end of the inner curved needle preferably defines a ramp to engagingly guide the suture.

In one embodiment, the handheld medical suturing device includes a single length of suture, where a first portion of the single length of suture is disposed around the at least one spool, where a second portion of the single length of suture is disposed along the curved suturing needle assembly, where a third portion of the single length of suture is disposed inside the bobbin carrier cavity, and where a fourth portion of the single length of suture is disposed around the bobbin.

In another one embodiment, the handheld medical suturing device includes a single length of suture, where a first portion of the single length of suture is disposed around the at least one spool, where a second portion of the single length of suture is disposed within the shroud of the curved suturing needle assembly, where a third portion of the single length of suture is disposed inside the bobbin carrier cavity, and where a fourth portion of the single length of suture is disposed around the bobbin.

In one embodiment, the curved suturing needle assembly includes a single curved needle having an inner surface which defines an annular groove. The single curved needle has a plurality of containment bridges annularly disposed along the annular groove of the inner surface. The plurality of containment bridges contain the suture within the annular groove.

In another embodiment, the curved suturing needle assembly includes a single curved needle that is a hollow needle, and suture is threaded through the hollow needle. In such embodiment, the curved suturing needle assembly includes a curved hollow needle body having a proximal end opposite a distal end. The proximal end of the curved hollow needle body defines a first orifice. The distal end of the curved hollow needle body defines a second orifice. The curved hollow needle body defines a passage therethrough between the first orifice and the second orifice, and the suture is threaded through the first orifice, through the passage, and through the second orifice.

As further embodied and described herein, there is provided a curved suturing needle assembly having an outer curved needle fixed to an inner curved needle. The outer curved needle has a proximal end opposite a distal end. The inner curved needle has a proximal end opposite a distal end.

In an embodiment, the curved suturing needle assembly includes a shroud disposed along an outer surface of the outer curved needle and the inner curved needle. The shroud forms a passage for the suture. Preferably, the shroud comprises a pointed cover encapsulating the distal end of the outer curved needle.

In yet another embodiment, the curved suturing needle assembly includes an elbow guide disposed within the pointed cover of the shroud. The elbow guide is configured to engage and guide the suture when the suture extends outward from the distal end of the inner curved needle. The distal of the inner curved needle preferably defines a ramp adapted to engagingly guide the suture.

In another embodiment, a suturing device is described. The suturing device may include an elongated body having a proximal end opposite a distal end, the elongated body defining a longitudinal axis. The device may further include a curved needle assembly arranged at the distal end of the elongated body and movable between at least an extended position and a retracted position. The curved needle assembly may be configured, in the extended position, to advance a first portion of a suture along a suture path through tissue, and may be configured, in the retracted position, to retract the first portion of the suture along the suture path. The device may further include a control assembly arranged at the proximal end of the elongated body. The control assembly may be configured to move the curved needle assembly.

The suturing device may further include a bobbin carrier arranged at the distal end of the elongated body. The bobbin carrier may be aligned with the curved needle assembly along the longitudinal axis. The suturing device may further include a bobbin positioned within the bobbin carrier and configured to carry a second portion of the suture. The suturing device may further include a movable arm operably coupled to the bobbin and configured to move the bobbin relative to the bobbin carrier.

The control assembly may be configured to move at least one of the curved needle assembly and the bobbin so as to form a locking stitch in the tissue between the first portion of the suture and the second portion of the suture.

The curved needle assembly moving to the extended position may further include forming a bow with the first portion of the suture. The locking stitch may be formed by moving the second portion of the suture via the bobbin through the bow and moving the curved needle assembly to the retracted position.

In the extended position, a curved needle of the curved needle assembly may be advanced part way through the tissue or in some cases entirely through the tissue.

The bobbin carrier may define an opening at a distal end, the opening configured to receive the tissue.

The control assembly may include at least a first actuator configured to move the curved needle assembly, and a second actuator configured to move the bobbin via the movable arm.

The bobbin carrier may define a groove and at least a portion of the movable arm may be configured to travel along the groove to move the bobbin relative to the bobbin carrier.

The suturing device may further include a magnet arranged on the movable arm. The magnet may be magnetically coupled with at least a portion of the bobbin such that movement of the movable arm causes corresponding movement of the bobbin relative to the bobbin carrier.

The portion of the bobbin magnetically coupled to the magnet may be a ferromagnetic ring arranged around at least a portion of the bobbin.

The suturing device may further include a scotch yoke mechanism operably coupled to the movable arm, the scotch yoke mechanism comprising a yoke and a gear set configured to convert movement generated by actuation of the control assembly into movement of the movable arm.

The bobbin carrier may be fixed relative to the elongated body, and the bobbin may be movable within the bobbin carrier.

The suturing device may further include a bobbin capsule positioned within the bobbin carrier. The bobbin capsule may be configured to retain the bobbin therein.

The curved needle assembly may include an arm coupled with the bobbin carrier at a fulcrum point. Movement of the curved needle assembly between the retracted position and the extended position may be defined by pivoting of the arm about the fulcrum point.

The control assembly may include a robot.

The suturing device may be adapted for use with a laparoscope or arthroscope.

In another embodiment, a method of forming a locking stitch in tissue with a handheld suturing device is described. The method may include positioning a curved needle assembly adjacent to tissue, and actuating a control assembly arranged to cause the curved needle assembly to move to an extended position and to advance a first portion of a suture along a suture path through the tissue. The curved needle assembly may be arranged at a distal end of an elongated body and the control assembly may be arranged at a proximal end of the elongated body. The method may further include actuating the control assembly to cause the curved needle assembly to move to a retracted position, thereby retracting the first portion of the suture along the suture path and securing the suture at the tissue.

The method may may further include actuating the control assembly to move a bobbin relative to a bobbin carrier arranged at the distal end of the elongated body. Moving the curved needle assembly to the extended position may further include forming a bow with the first portion of the suture and movement of the bobbin may cause a second portion of the suture carried by the bobbin to move through the bow. The method may further include actuating the control assembly to cause the curved needle assembly to move to a retracted position, thereby retracting the first portion of the suture along the suture path and forming a locking stich at the tissue with the second portion of the suture.

In the extended position, a curved needle of the curved needle assembly is advanced part way through the tissue.

The method may further include positioning at least a portion of the tissue within an opening defined at a distal end of the bobbin carrier.

Actuating the control assembly may cause the curved needle assembly to move to the extended position includes actuating a first actuator of the control assembly.

Actuating the control assembly to move the bobbin relative to the bobbin carrier may include actuating a second actuator of the control assembly.

Moving the bobbin relative to the bobbin carrier may include moving at least a portion of a movable arm along a groove defined by the bobbin carrier. The movable arm may be operably coupled with the bobbin.

Moving the bobbin relative to the bobbin carrier may further include magnetically coupling a magnet on the movable arm with at least a portion of the bobbin, such that movement of the movable arm causes corresponding movement of the bobbin.

The movable arm may be arranged outside a wall of the bobbin carrier such that the bobbin is moved through the wall of the bobbin carrier by the magnetic coupling.

The bobbin carrier may remain fixed relative to the elongated body while the bobbin is moved within the bobbin carrier.

Moving the bobbin relative to the bobbin carrier may include actuating at least one of a scotch yoke mechanism operably coupled to the movable arm, or a ball screw actuator operably coupled to the movable arm.

The actuation of the control assembly may include a robot.

The method may further include forming a plurality of locking stitches in tissue in one or more of the following: repair of hernias with or without mesh reinforcement, repair of diaphragmatic defects or ruptures, closure or reinforcement of gastrointestinal tissue, reinforcement of staple lines, repair of tendons, ligaments, or other soft tissues, repair of rotator cuff injuries, repair of thoracic structures, and vascular repair.

The tissue may be human tissue.

The tissue may be animal tissue.

In some embodiments, a laparoscope may include an assembly comprising a suturing device as described above and below. In some embodiments, laparoscopic surgery may include the use of a laparoscope comprising a suturing device described herein. In still other embodiments, one or more suturing devices as described herein may be used with one or more robots, wherein the one or more robots actuate the one or more suturing devices to suture tissue. It should also be noted that the suturing device as disclosed herein may be used to suture tissue of human patients, as well as for veterinary uses, as well as with wildlife and/or animals in zoos.

These and other features, aspects, and advantages of the present invention will become better understood with reference to the following description and appended claims.

### BRIEF DESCRIPTION OF THE DRAWINGS

Illustrative embodiments of the present invention are described herein with reference to the accompanying drawings, in which:

FIG. 1 is a partial cutaway cross-sectional view of an exemplary handheld medical suturing device in a rest position and showing an exemplary tissue, in accordance with embodiments of the invention;

FIG. 2 is another partial cutaway cross-sectional view of the exemplary handheld medical suturing device showing additional embodiments in a rest position and showing an exemplary tissue, in accordance with embodiments of the invention;

FIG. 3 is another partial cutaway cross-sectional view of the exemplary handheld medical suturing device in a fully extended position and showing an exemplary tissue, in accordance with embodiments of the invention;

FIG. 4 is a partial cutaway perspective view of embodiments of a fixed bobbin channel, a bobbin carrier and a bobbin in a rest position, and a curved suturing needle assembly, of the handheld medical suturing device, in accordance with embodiments of the invention;

FIG. 5 is a partial cutaway perspective view of embodiments of a fixed bobbin channel, a bobbin carrier and a bobbin in an actuated position, and a curved suturing needle assembly, of the handheld medical suturing device, in accordance with embodiments of the invention;

FIG. 6 is a partial cutaway perspective view of a curved suturing needle assembly showing an exemplary user's hand grasping the curved suturing needle assembly, in accordance with embodiments of the invention;

FIG. 7 is a rear partial cutaway cross-sectional view of embodiments of a fixed bobbin channel, a bobbin carrier, a mutilated spur gear engaging a rack, a first thumb lever, a second thumb lever and a respective first drive gear and a respective second drive gear, of the handheld medical suturing device, in accordance with embodiments of the invention;

FIG. 8 is a top partial cutaway cross-sectional view of a handheld medical suturing device showing a first thumb lever in a first thumb lever first position, in accordance with embodiments of the invention;

FIG. 9 is a bottom partial cutaway cross-sectional view of a handheld medical suturing device showing a first thumb lever in a first thumb lever first position, in accordance with embodiments of the invention;

FIG. 10 is a bottom partial cutaway cross-sectional view of a handheld medical suturing device showing a first thumb lever in a first thumb lever second position, in accordance with embodiments of the invention;

FIG. 11 is rear partial cutaway cross-sectional view of embodiments of a handheld medical suturing device, showing the first thumb lever in a first thumb lever first position, and showing a second thumb lever in a second thumb lever first position, in accordance with embodiments of the invention;

FIG. 12 is rear partial cutaway cross-sectional view of embodiments of a handheld medical suturing device, showing the first thumb lever in a first thumb lever second position, and showing the second thumb lever in a second thumb lever second position, in accordance with embodiments of the invention;

FIG. 13 is a cross sectional view along the cutting view 13-13 of FIG. 1, of embodiments of a handheld medical suturing device, in accordance with embodiments of the invention;

FIG. 14 is a perspective view of a spool of a handheld medical suturing device, in accordance with embodiments of the invention;

FIG. 15 is a partial cutaway cross-sectional view of a handheld medical suturing device, showing a curved needle suturing assembly in a retracted position, in accordance with embodiments of the invention;

FIG. 16 is a cross sectional view along the cutting view 16-16 of FIG. 15 of an actuating arm of a handheld medical suturing device, in accordance with embodiments of the invention;

FIG. 17 is a partial cutaway cross-sectional view of embodiments of a handheld medical suturing device, showing a curved needle suturing assembly in a fully extended position, in accordance with embodiments of the invention;

FIG. 18 is a side elevation, partial cutaway, view of a handheld medical suturing device, in accordance with embodiments of the invention;

FIG. 19 is a top left perspective partial cutaway view of a handheld medical suturing device, in accordance with embodiments of the invention;

FIG. 20 is a partial cutaway cross-sectional view a curved needle suturing assembly, in accordance with embodiments of the invention;

FIG. 21 is a cross sectional view along the cutting view 21-21 of FIG. 20, in accordance with embodiments of the invention;

FIG. 22 is a cross sectional view along the cutting view 22-22 of FIG. 20, in accordance with embodiments of the invention;

FIG. 23 is a partial cutaway cross-sectional view of a curved needle suturing assembly, in accordance with embodiments of the invention;

FIG. 24 is a perspective partial cutaway view of a curved needle suturing assembly, in accordance with embodiments of the invention;

FIG. 25 is a perspective partial cutaway view of embodiments of a curved needle suturing assembly, in accordance with embodiments of the invention;

FIG. 26 is a perspective partial cutaway view of an elbow of a curved needle suturing assembly, in accordance with embodiments of the invention;

FIG. 27 is a bottom partial cutaway cross-sectional view of a curved needle suturing assembly, in accordance with embodiments of the invention;

FIG. 28 is a partial cutaway cross-sectional view a curved needle suturing assembly, in accordance with embodiments of the invention;

FIG. 29 is a side elevation partial cutaway view of a shroud of a curved needle suturing assembly, in accordance with embodiments of the invention;

FIG. 30 is a cross sectional view along the cutting view 30-30 of FIG. 29, in accordance with embodiments of the invention;

FIG. 31 is a cross sectional view along the cutting view 31-31 of FIG. 20, in accordance with embodiments of the invention;

FIG. 32 is a top partial cutaway view of a shroud of a curved needle suturing assembly, in accordance with embodiments of the invention;

FIG. 33 is partial cutaway cross-sectional view of a handheld medical suturing device, showing a curved needle suturing assembly in a retracted position, in accordance with embodiments of the invention;

FIG. 34 is a bottom partial cutaway cross-sectional view of a curved needle suturing assembly, in accordance with embodiments of the invention;

FIG. 35 is a cross sectional view along the cutting view 35-35 of FIG. 34, in accordance with embodiments of the invention;

FIG. 36 is a partial cutaway cross-sectional view of a handheld medical suturing device, showing a curved needle suturing assembly in a retracted position, in accordance with embodiments of the invention;

FIG. 37 is a cross sectional view along the cutting view 37-37 of FIG. 36, in accordance with embodiments of the invention;

FIG. 38 is a view of a partial cutaway cross-sectional view of an exemplary bent constrained rigid body of a handheld medical suturing device, in accordance with embodiments of the invention;

FIG. 39 is a top partial cutaway cross-sectional view of a curved needle suturing assembly, in accordance with embodiments of the invention;

FIG. 40 is a partial cutaway view of a spool, a spring biasing member, a curved suturing needle assembly, and a bobbin of a handheld medical suturing device, in accordance with embodiments of the invention;

FIG. 41 is a partial cutaway view of a spool, a spring biasing member, a curved suturing needle assembly, a bobbin of a handheld medical suturing device, with an exemplary tissue, in accordance with embodiments of the invention;

FIG. 42 is partial cutaway view of a curved suturing needle assembly, a suture, and a bobbin of a handheld medical suturing device, with the curved suturing needle assembly positioned to penetrate an exemplary tissue, in accordance with embodiments of the invention;

FIG. 43 is a partial cutaway view of a curved suturing needle assembly, a suture, and a bobbin of a handheld medical suturing device, with the bobbin in a rest position and with the curved suturing needle assembly in a fully extended position as penetrated through exemplary tissue, in accordance with embodiments of the invention;

FIG. 44 is a partial cutaway view of a curved suturing needle assembly, a suture, and a bobbin of a handheld medical suturing device, with the bobbin in an actuated position and with the curved suturing needle assembly in a fully extended position as penetrated through exemplary tissue, in accordance with embodiments of the invention;

FIG. 45 is an enlarged view of a portion the suture of FIG. 44, illustrating a crossing of the suture to form a locking of the suture, in accordance with embodiments of the invention;

FIG. 46 is a partial cutaway view of a curved suturing needle assembly, a suture, and a bobbin of a handheld medical suturing device, illustrating a locking of the suture, in accordance with embodiments of the invention;

FIG. 47 is an enlarged partial cutaway view of a curved suturing needle assembly, a suture, and a bobbin of a handheld medical suturing device, illustrating a locking of the suture, in accordance with embodiments of the invention;

FIG. 48 is a partial cutaway view of a curved suturing needle assembly, a suture, and a bobbin of a handheld medical suturing device, illustrating a locking of the suture, with two pieces of exemplary tissue, in accordance with embodiments of the invention;

FIG. 49 is an enlarged partial cutaway view of a curved suturing needle assembly, a suture, and a bobbin of a handheld medical suturing device, illustrating a locking of the suture, with two pieces of exemplary tissue, in accordance with embodiments of the invention;

FIG. 50 is a partial cutaway view of a curved suturing needle assembly, a suture, and a bobbin of a handheld medical suturing device, with the bobbin in a rest position, illustrating a locking of the suture, illustrating a first piece and a second piece of exemplary tissue, and with the curved suturing needle assembly positioned to penetrate the second exemplary tissue, in accordance with embodiments of the invention;

FIG. 51 is a partial cutaway view of a view of a curved suturing needle assembly of a handheld medical suturing device forming a bow with a suture, in accordance with embodiments of the invention;

FIG. 52 is a partial cutaway view of a view of a curved suturing needle assembly of a handheld medical suturing device after forming a bow with a suture, with the curved suturing needle assembly in a retracted position, in accordance with embodiments of the invention;

FIG. 53 is a partial cutaway view of a view of a curved suturing needle assembly of a handheld medical suturing device after forming a bow with a suture, with the curved suturing needle assembly in a retracted position, in a penetrated position through a second piece of tissue, and in another rest position, in accordance with embodiments of the invention;

FIG. 54 is a partial cutaway view of a view of a handle body and a handle lever of a handheld medical suturing device, illustrating at least one locking pin aligned with a channel defined by the handle lever, in accordance with embodiments of the invention;

FIG. 55 is a partial cutaway view of a view of a handle body and a handle lever of a handheld medical suturing device, illustrating at least one locking pin penetrated within a channel defined by the handle lever, in accordance with embodiments of the invention;

FIG. 56 is a side partial cutaway view of another exemplary handheld medical suturing device in a rest position and showing an exemplary tissue, in accordance with embodiments of the invention;

FIG. 57 is a side view, partly cutaway, of the exemplary handheld medical suturing device of FIG. 56 in a fully extended position and showing the exemplary tissue of FIG. 56, in accordance with embodiments of the invention;

FIG. 58 illustrates a perspective view of another medical suturing device, in accordance with embodiments of the invention;

FIG. 59 illustrates a side view of the suturing device, in accordance with embodiments of the invention;

FIG. 60 illustrates a side view of a distal portion of the suturing device with a curved needle assembly in a retracted position, in accordance with embodiments of the invention;

FIG. 61 illustrates a side view of the distal portion of the suturing device of FIG. 60 with the curved needle assembly in an extended position, in accordance with embodiments of the invention;

FIG. 62 illustrates a side view of the distal portion of the suturing device including a bell crank actuation assembly, in accordance with embodiments of the invention;

FIG. 63 illustrates a side view of the distal portion of the suturing device including another bell crank actuation assembly, in accordance with embodiments of the invention;

FIG. 64 illustrates a side view of the distal portion of the suturing device including a linear actuator assembly, in accordance with embodiments of the invention;

FIG. 65 illustrates a side view of the distal portion of the suturing device including another linear actuator assembly, in accordance with embodiments of the invention;

FIG. 66 illustrates a side view of the distal portion of the suturing device including a scotch yoke actuator assembly, in accordance with embodiments of the invention; and

FIG. 67 illustrates a side view of the distal portion of the suturing device including another scotch yoke actuator assembly, in accordance with embodiments of the invention.

The figures depict various embodiments for purposes of illustration only. One skilled in the art will readily recognize from the following discussion that alternative embodiments of the structures and methods illustrated herein may be employed without departing from the principles of the embodiments described herein.

### DETAILED DESCRIPTION

For a further understanding of the nature and function of the embodiments, reference should be made to the following detailed description. Detailed descriptions of the embodiments are provided herein, as well as, the best mode of carrying out and employing the present invention. It will be readily appreciated that the embodiments are well adapted to carry out and obtain the ends and features mentioned as well as those inherent herein. It is to be understood, however, that the present invention may be embodied in various forms. Therefore, persons of ordinary skill in the art will realize that the following disclosure is illustrative only and not in any way limiting, as the specific details disclosed herein provide a basis for the claims and a representative basis for teaching to employ the present invention in virtually any appropriately detailed system, structure or manner. It should be understood that the devices, materials, methods, procedures, and techniques described herein are presently representative of various embodiments. Other embodiments of the disclosure will readily suggest themselves to such skilled persons having the benefit of this disclosure.

For purposes of clarity and orientation with respect to a person, referred to herein as a user, it is noted that a transverse (also known as axial or horizontal) plane is an X′Z plane, parallel to the ground. A frontal (also known as coronal) plane is a YX plane, perpendicular to the ground, which (in humans) separates the anterior from the posterior, the front from the back, the ventral from the dorsal. A sagittal (also known as lateral) plane is an XY plane, perpendicular to the ground, which separates left from right. Objects are coplanar if they all lie in the same plane. For example, one axis is coplanar with another axis when the two axes lie in the same plane.

As used herein, “axis” means a real or imaginary straight line about which a three-dimensional body is symmetrical. A “vertical axis” means an axis perpendicular to the ground (or put another way, an axis extending upwardly and downwardly). A “horizontal axis” means an axis parallel to the ground.

As used herein, homogeneous is defined as the same in all locations, and a homogeneous material is a material of uniform composition throughout that cannot be mechanically separated into different materials. Examples of “homogeneous materials” are certain types of plastics, ceramics, glass, metals, alloys, paper, board, resins, and coatings.

Reference will now be made in detail to the present embodiments of the invention, examples of which are illustrated in the accompanying drawings. Wherever possible, the same reference numerals are used in the drawings and the description to refer to the same or like parts.

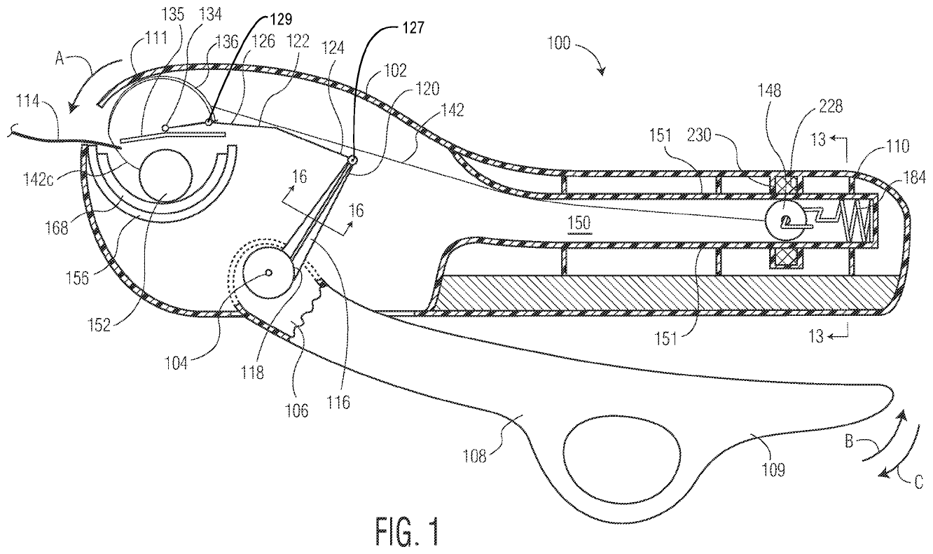

Referring initially to FIGS. 1-3, the basic constructional details and principles of operation of one embodiment of a handheld medical suturing device 100 according to an embodiment of the present invention will be discussed. The handheld medical suturing device 100 preferably has a handle body 102 having a hinge 104 operatively connected to a distal end 106 of a handle lever 108. The handle lever is adapted to be grasped by a hand of a user. The handle body 102 has proximal end 110 adapted to be grasped by a user and a distal end 111 which defines an opening 112 adapted to receive a tissue 114, as exemplified in FIGS. 1-3 and 19.

In an embodiment, the handheld medical suturing device 100 includes an actuating arm 116 disposed within the handle body 102. The actuating arm 116 has a proximal end 118 and a distal end 120, and the proximal end 118 of the actuating arm 116 is fixed to the distal end 106 of the handle lever 108, as illustrated in FIG. 1.

As seen in FIGS. 1-3, in one embodiment, the handheld medical suturing device includes a bent constrained rigid body 122, which has a proximal end 124 and a distal end 126. The proximal end 124 of the bent constrained rigid body 122 is operatively connected to the actuating arm 126 at a moveable joint 127 (FIG. 38). The distal end 126 of the bent constrained rigid body 122 is operatively connected to a pivoting radial arm 128 (not shown), preferably at a moveable joint 129 (FIG. 38). For purposes of clarity, in the partial cutaway top view of FIG. 38, the curved suturing needle assembly 136 obstructs the view of the pivoting radial arm 128 in FIG. 38.

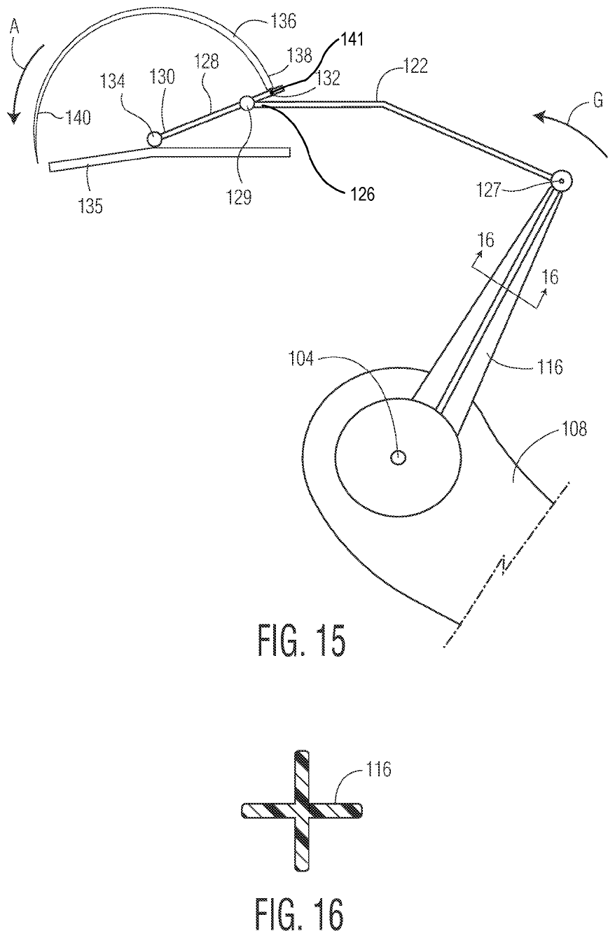

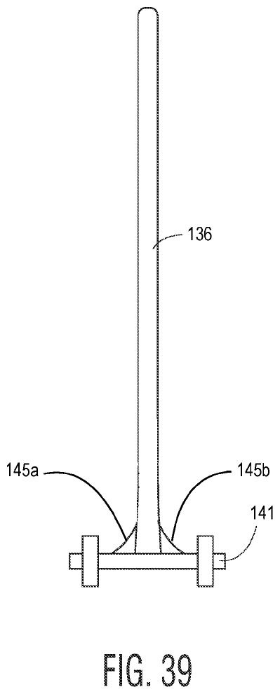

FIGS. 15-17 illustrate enlarged views of the actuating arm 116 operatively connected to the bent constrained rigid body 122, which is operatively connected to the pivoting radial arm 128, and FIGS. 38-39 illustrate enlarged views of the moveable joints 127, 129. However, the pivoting radial arm 128 is clearly illustrated in at least FIGS. 1-3, 15, 17, and 33.

The pivoting radial arm 128 has a proximal end 130 opposite a distal end 132, as exemplified in FIGS. 1 and 15. Referring to FIG. 15, the proximal end 130 of the pivoting radial arm 128 is operatively connected to a fulcrum point 134 that is disposed within the handle body 102, preferably at a position just above a transverse separator wall 135 that is fixed within the handle body 102, preferably as illustrated in at least FIGS. 1-3. The distal end 132 of the pivoting radial arm 128 is operatively connected to the distal end 126 of the bent constrained rigid body 122.

Referring initially to FIGS. 1-5 and 15-17, the handheld medical suturing device 100 includes a curved suturing needle assembly 136 disposed within the handle body 102. The curved suturing needle assembly 136 has a proximal end 138 and a distal end 140. The proximal end 138 is operatively connected to the distal end 132 of the pivoting radial arm 130, as exemplified in FIG. 15.

Preferably, the proximal end 138 of the curved suturing needle assembly 136 is operatively connected to the distal end 132 of the pivoting radial arm 128 at a needle fulcrum point 141, as illustrated in FIGS. 15, 17, and 39, for example. The fulcrum point 141 preferably has an axis that is both transverse to the pivoting radial arm 128 and to the curved suturing needle assembly 136. As illustrated in FIG. 39, in an embodiment, the curved suturing needle assembly 136 includes lateral supports 145a, 145b each fixed to the proximal end 138 of the curved suturing needle assembly 136 and the needle fulcrum point 141, to provide additional rigidity.

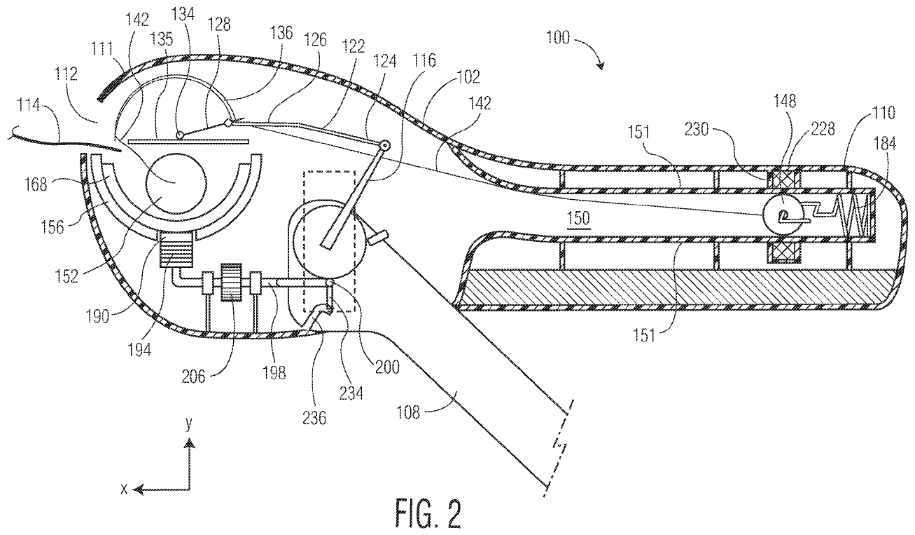

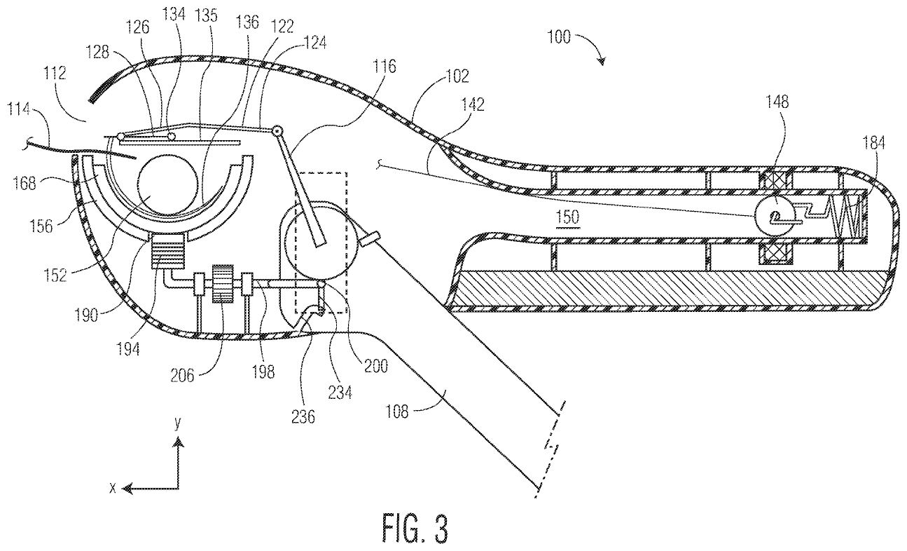

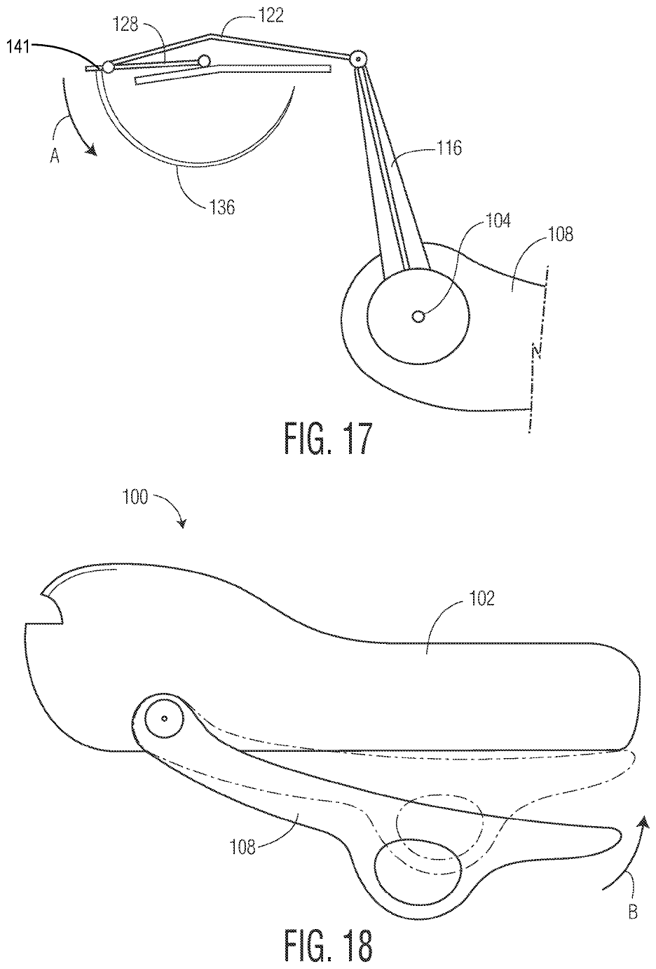

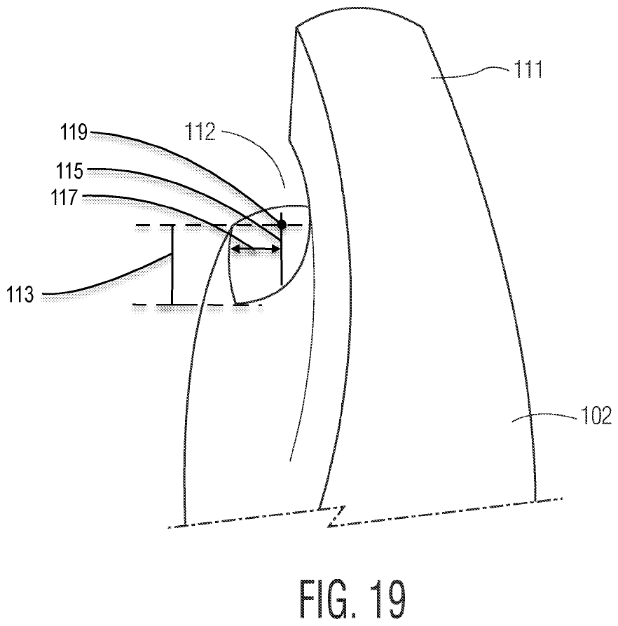

The curved suturing needle assembly 136 is adapted to advance a suture 142 through the tissue 144, in the direction of Arrow A in FIG. 1, by a rotation of the actuating arm 116 and respective rotation of the bent constrained rigid body 122, in the direction of Arrow G in FIG. 15, along an XY plane (FIG. 2) that is substantially perpendicular to the tissue 114, which is substantially on an transverse plane, which is an X′Z plane. Such rotation of the actuating arm 116 and respective rotation of the bent constrained rigid body 122 (in the direction of Arrow G) rotates the curved suturing needle assembly 136 through the tissue 114 between a retracted position (as illustrated in FIGS. 1 and 15) and a fully extended position (as illustrated in FIGS. 3 and 17), because the pivoting radial arm 128 pivots around the fulcrum point 134, upon a pivoting of the handle lever 108 (in the direction of Arrow Bin FIG. 18) relative to the handle body 102 from an open position (as illustrated in FIGS. 1, 2, and 15) to a squeezed position (as illustrated in FIGS. 3 and 17).

As illustrated in FIGS. 1-4 and 19, after the tissue 114 is inserted into the opening 112, the curved suturing needle assembly 136 can be rotated, and thus actuated and advanced, through the tissue 114, in the direction of Arrow A in FIG. 1, while the curved suturing needle assembly 136 remains fully disposed within and concealed by the handle body 102, thus overcoming the limitations of conventional medical devices, where the suturing needle is exposed, as such exposure gives rise to risk to the surgeon. As can be appreciated from the present disclosure, the embodiment of the disclosed invention provide a much safer handheld medical suturing device 100, because the curved suturing needle assembly 136 is guarded by the by the handle body 102, even when the curved suturing needle assembly 136 is actuated and advanced through the tissue 114 and/or tissues 114a, 114b. In other words, by the handle body 102 and its respective opening 112 mitigate risk of exposure of, and accidental contact between, the suturing needle and unintended objects such as a surgeon's hand. As such, it can be appreciated that, since the curved needle suturing assembly 136 is fully contained within the handle body 102 of the handheld medical suturing device 100, the handheld medical suturing device 100 greatly diminishes the chance for accidental puncture wounds to the surgeon and/or assistant, and thus, the disclosed invention reduces the chance for transmission of disease, such as hepatitis.

In an embodiment, referring to FIGS. 1-5, the handheld medical suturing device 100 further includes at least one spool 148 of suture 142 disposed within an inner cavity passageway 150 defined by the proximal end 110 of the handle body 102. The handle body 102 preferably comprises an internal rectangular tube 151 of walls that define the inner cavity passageway 150. The suture 142 is threaded from the at least one spool 148 through the inner cavity passageway 150 of the handle body 102 to the curved suturing needle assembly 136. The suture 142 is further threaded from the curved suturing needle assembly 136 to a bobbin 152 that is disposed within the distal end 111 of the handle body 102 of the handheld medical suturing device 100. The at least one spool 148 is configured to dispense the suture to the curved suturing needle assembly 136 upon the rotation of the curved suturing needle assembly from the retracted position (illustrated in FIGS. 1 and 15) to the fully extended position (illustrated in FIGS. 3, 17 and 43).

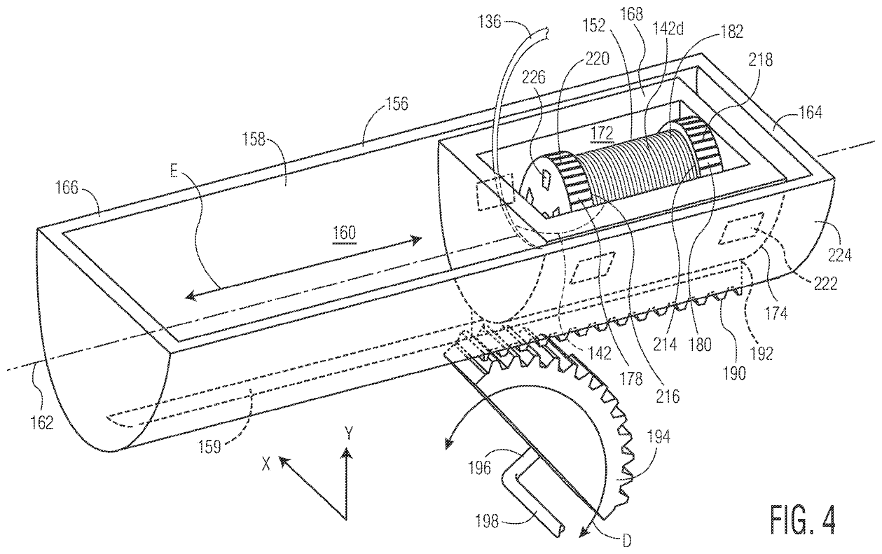

In yet another embodiment, the handheld medical suturing device 100 further includes a fixed bobbin channel 156 that preferably has a semi-annular body 158 as exemplified in FIG. 4, which forms a bobbin channel cavity 160 disposed within the distal end 111 of the handle body 102. The semi-annular body 158 defines an elongated slot 159 therethrough, preferably located at the bottom of the semi-annular body 158 of the fixed bobbin channel 156. The semi-annular body 158 has a proximal end 164 opposite a distal end 166, and the bobbin channel cavity 160 has a central axis 162 that is perpendicular to the XY plane (FIGS. 2-4).

The handheld medical suturing device 100 preferably includes a bobbin carrier 168 that is movably fitted within the bobbin channel cavity 160 of the fixed bobbin channel 156. The bobbin carrier 168 preferably has a semi-annular body 170 that forms a bobbin carrier cavity 172 that is sized and configured to closely receive the curved suturing needle assembly 136 in the fully extended position (illustrated in FIGS. 3 and 17). The curved suturing needle assembly 136 is illustrated in a partially extended position in FIG. 4.

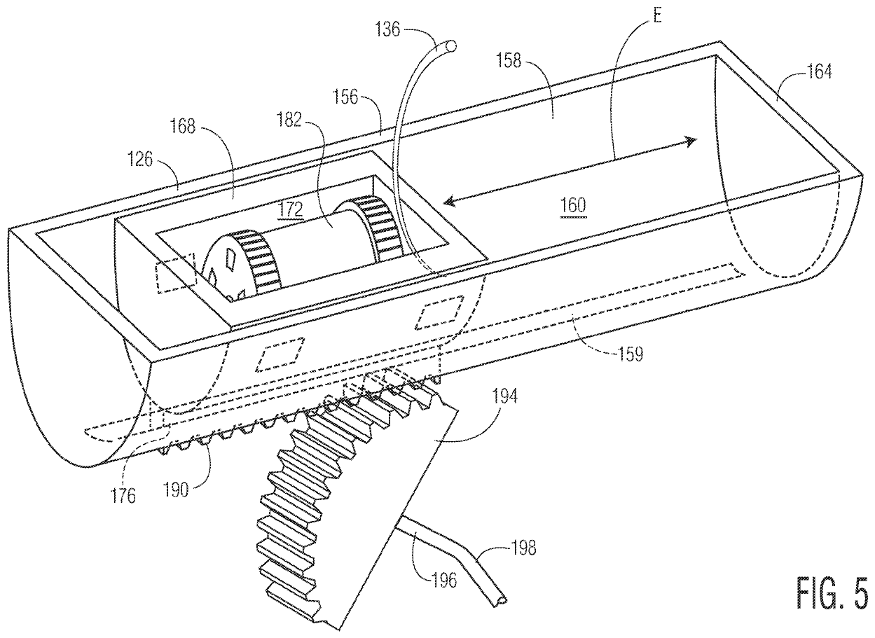

As illustrated in FIGS. 4-5, the semi-annular body of the bobbin carrier 170 closely aligns with the semi-annular body 158 of the fixed bobbin channel 156.

The bobbin carrier 168 is adapted for movement of the bobbin 152 along the central axis 162 across the XY plane. The bobbin carrier 168 moves between a rest position 174 (FIG. 4) and an actuated position 176 (FIG. 5), as the bobbin 152 is rotatably disposed within the bobbin carrier cavity 172 of the bobbin carrier 168.

As exemplified in FIGS. 4-5, the bobbin carrier 168 of the handheld medical suturing device 100 comprises a rack 190 fixed to and disposed along a bottom of the bobbin carrier 168. Preferably, the rack 190 is preferably molded as part of the bobbin carrier 168. The rack 190 is operatively fitted within the elongated slot 159 of the bottom 192 of the fixed bobbin channel 156. As such, the rack 190 can slide back and forth along the elongated slot 159, in the direction of Arrow E.

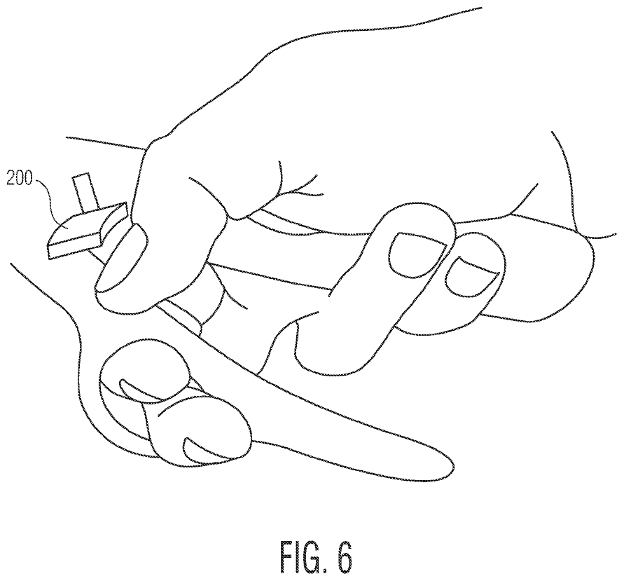

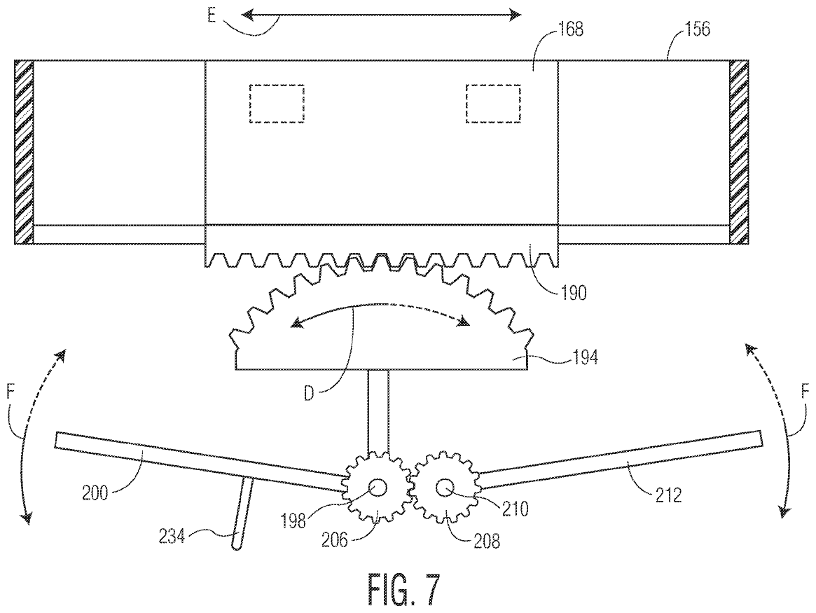

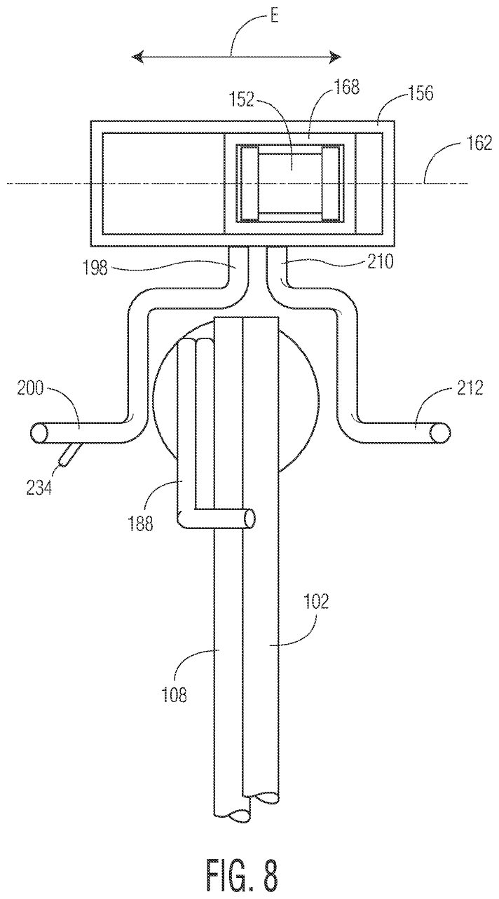

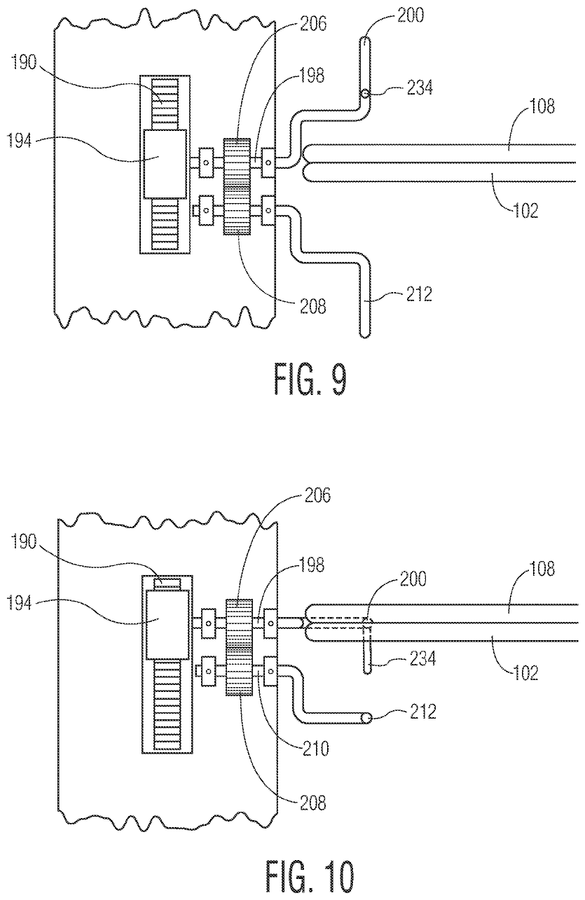

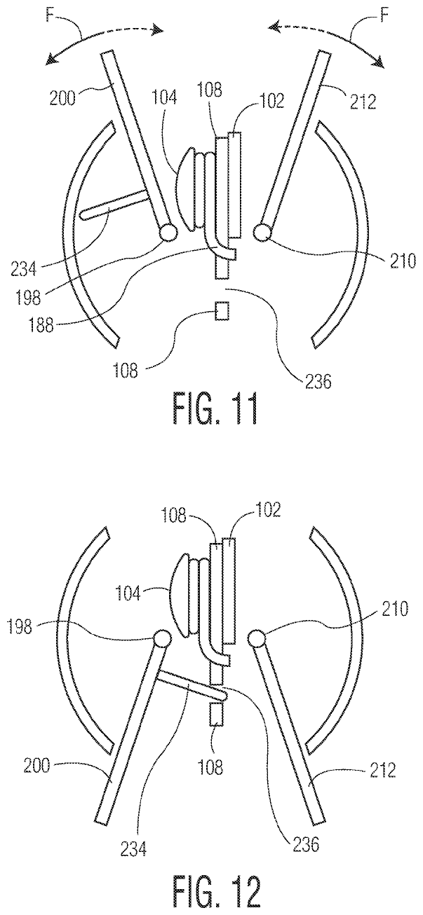

Referring to FIGS. 4-12, in an embodiment, the handheld medical suturing device 100 preferably includes a mutilated spur gear 194 that is fixed to a first drive axle 198 by an arm 196. The first drive axle 198 is connected to a first thumb lever 200 that extends outwardly of the handle body 102, as shown in FIG. 6, for example. A rotation of the first thumb lever 200 (in the direction of Arrow F in FIG. 11) causes the first drive axle to rotate the mutilated spur gear 194, as exemplified in FIG. 7, from a first thumb lever first position, as illustrated in FIGS. 2, 7, 8, 9, and 11, to a first thumb lever second position, as illustrated in FIGS. 10 and 12.

Upon such rotation of the first thumb lever 200, the mutilated spur gear 194 operatively engages the rack 190 to translate and impart rotational motion, in the direction of Arrow D, of the mutilated spur gear 194 into linear motion, in the direction of Arrow E (FIGS. 4-5 and 7-8), of the rack 190 from the rest position 174 to the actuated position 176, upon the rotation, in the direction of Arrow F, of the first thumb lever 200 from a first thumb lever first position (as illustrated in FIGS. 2, 7, 8, 9, and 11) to a first thumb lever second position (as illustrated in FIGS. 10 and 12). The linear motion (Arrow E) of the rack 190 moves the bobbin carrier 168 and the bobbin 152 side to side in the direction of Arrow E within the fixed bobbin channel 156.

As exemplified in FIGS. 4-5, in an embodiment, the bobbin 152 of the handheld medical suturing device 100 has opposing hemispherical heads 214, 216 and a cylindrical body 182, which is preferably at least substantially cylindrical in shape, that serves as an axle disposed between such opposing hemispherical heads 214, 216. That axle contains the suture between the opposing hemispherical heads 214, 216.

In another embodiment, the head of the bobbin 152 is in the form of a hemispherical head, and a side wall of the hemispherical head of the bobbin 152 defines an orifice that is disposed at a central tip of the hemispherical head, where suture is dispensed from within the bobbin 152 through the orifice.

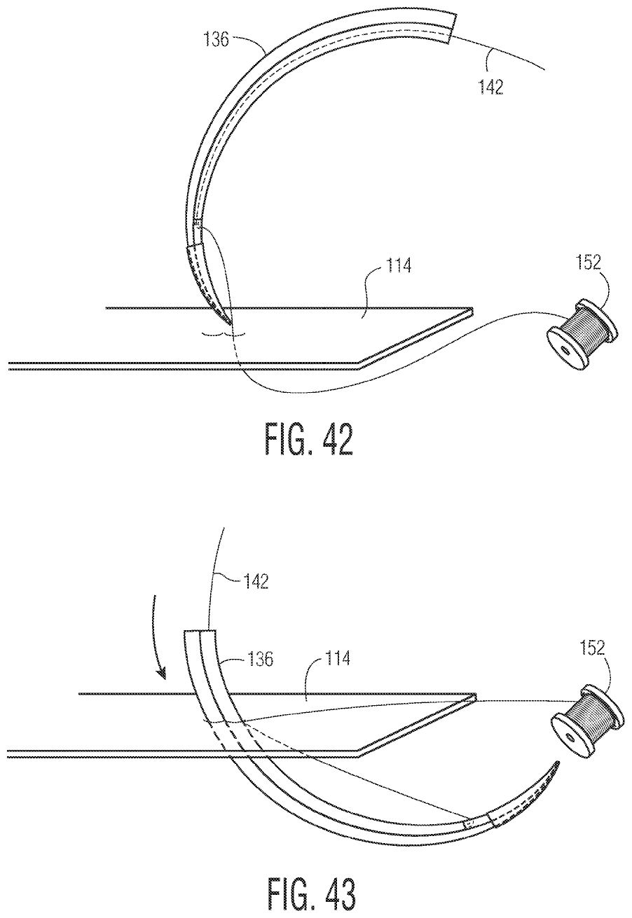

The substantially cylindrical body 182 releases the suture 142 therefrom upon the movement of the bobbin carrier 168 from the rest position 174 to the actuated position 176, in the direction of Arrow E, such that the bobbin 152 is moved across the XY plane, preferably when the curved needle suturing assembly 136 is in the fully extended position (FIGS. 3 and 17). The movement of the bobbin carrier 168 across the XY plane (FIGS. 2-4) moves the suture 142 through a bow 143 that is formed by the suture extending from the curved suturing assembly 136, as illustrated in FIGS. 42-45 and 52.

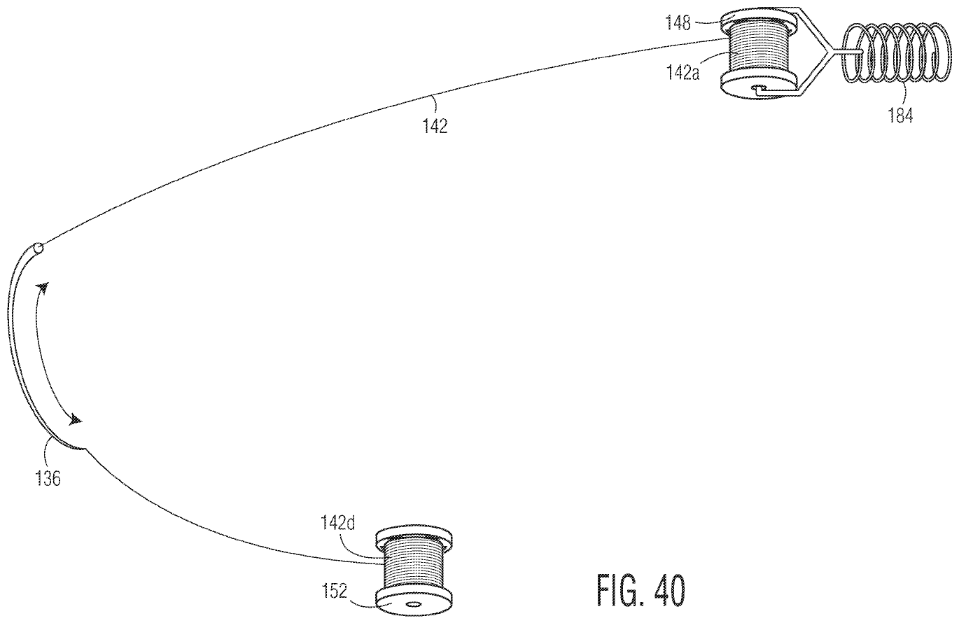

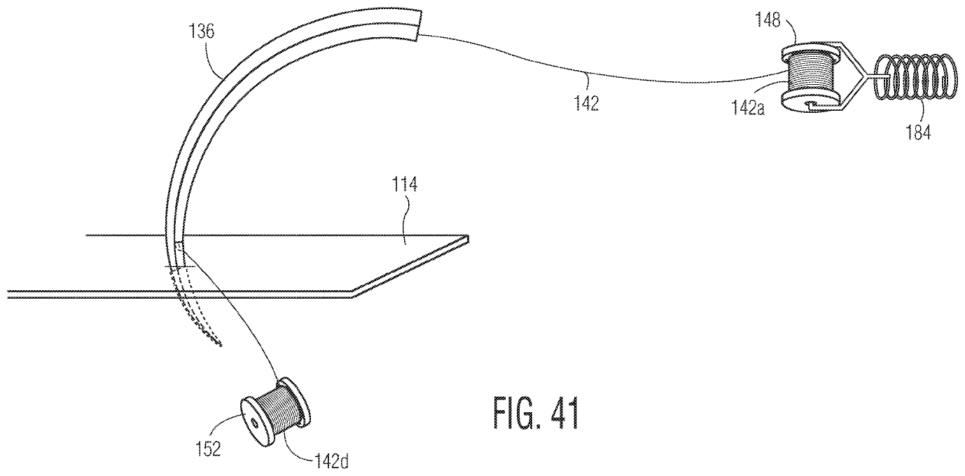

As illustrated in FIGS. 1-3, the handheld medical suturing device 100 preferably includes at least one spring biasing member 184 that is disposed within the inner cavity passageway 150 of the handle body 102. The spring biasing member 184 is preferably positioned between and connected to the at least one spool 148 and an inner wall 186 of the proximal end 110 of the handle body 102. The at least one biasing member 184 tensions the suture 142 between the at least one spool 148 and the bobbin 152, as exemplified in FIGS. 40-41.

Referring to FIGS. 1 and 11, the handheld medical suturing device 100 further includes at least one spring biasing member 188 that is disposed around the hinge 104 of the handheld medical suturing device 100. The at least one spring biasing member 188 is operatively connected to the handle body 102 and to the distal end 106 of the handle lever 108 at the hinge 104. The at least one biasing member 188 pivotally biases (the proximal end 109) of the handle lever 108 away from the proximal end 110 of the handle body 102 in the direction of Arrow C, illustrated in FIG. 1.

Referring to FIGS. 7-10, in an embodiment, the handheld medical suturing device 100 further comprises a first spur gear 206 that is concentrically disposed around and fixed to the first drive axle 198. The handheld medical suturing device 100 further comprises a second spur gear 208 concentrically disposed around and fixed to a second drive axle 210. The second drive axle 210 is connected to a second thumb lever 212, which extends outward of the handle body 102. The first spur gear 206 operatively engages the second spur gear 208. Similarly, the second spur gear 208 engages the first spur gear 206 to impart rotational movement, in the direction of Arrow Fin FIG. 7.

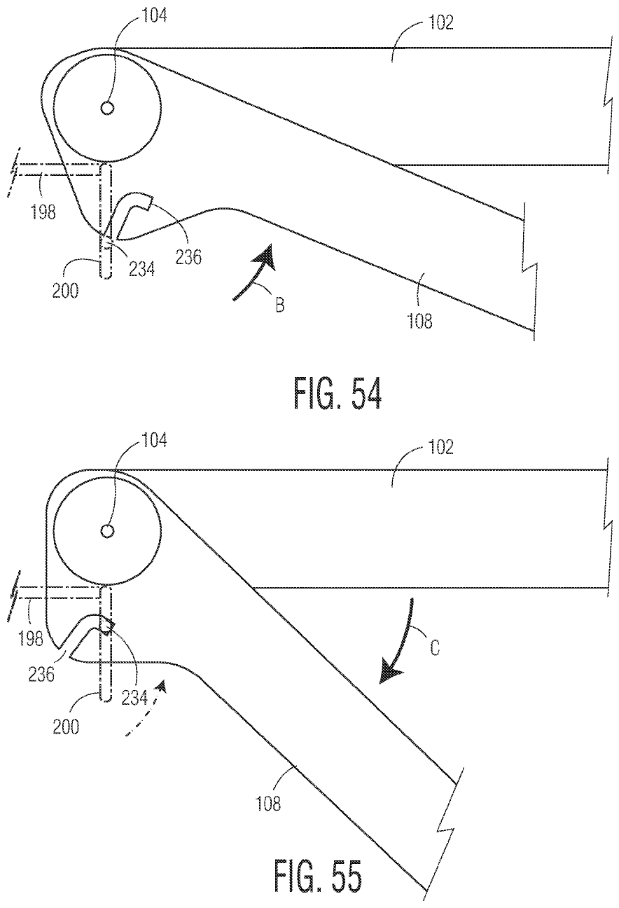

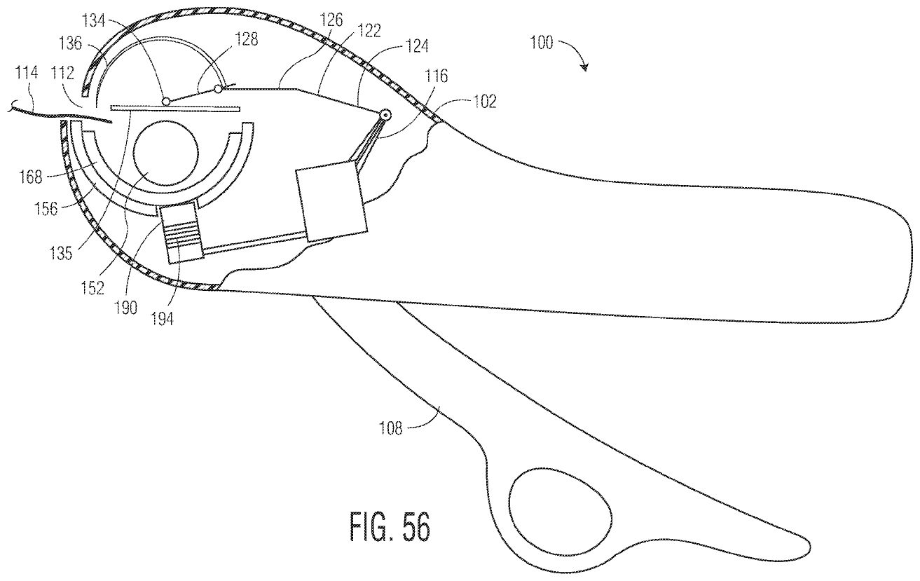

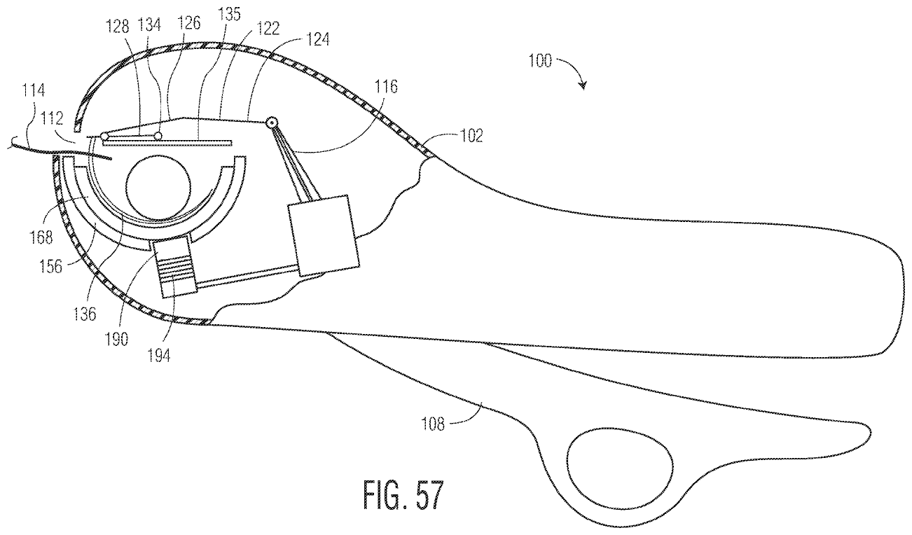

The handheld medical suturing device 100 preferably includes at least one locking pin 234 fixed to the first thumb lever 200. Preferably, the handle lever 108 defines a channel 236 (exemplified in FIGS. 2, 54 and 55), and when the handheld medical suturing device 100 is in the squeezed position (Arrow B of FIGS. 1 and 54) and in the actuated position (FIGS. 5 and 10), a downward rotation of the first thumb lever 200 (in the direction of solid Arrow Fin FIG. 11) causes the at least one locking pin 234 to align with that channel 236, as exemplified in FIG. 54, upon such downward rotation of the first thumb lever 200 from the first thumb lever first position, as illustrated in FIGS. 2, 7, 8, 9, and 11, to the first thumb lever second position, as illustrated in FIGS. 10 and 12. When the handle lever 108 is pivoted (re-opened) in the direction of Arrow C in FIGS. 1 and 55, the locking pin 234 penetrates and slides into the channel 236 of the handle body 108 into a locked position, as exemplified in FIG. 55.

Referring to FIGS. 12 and 54-55, such penetration of the locking pin 234 in the channel 236 of the handle body 108 obstructs and prevents a second pivoting (in the direction of Arrow B of FIGS. 1 and 54) of the handle lever 108 relative to the handle body 102 from the open position to the squeezed position, until after the thumb lever 200 has been rotated (dotted Arrow F of FIG. 11) upwardly so as to remove the locking pin 234 from the channel 236 of the handle body 108.

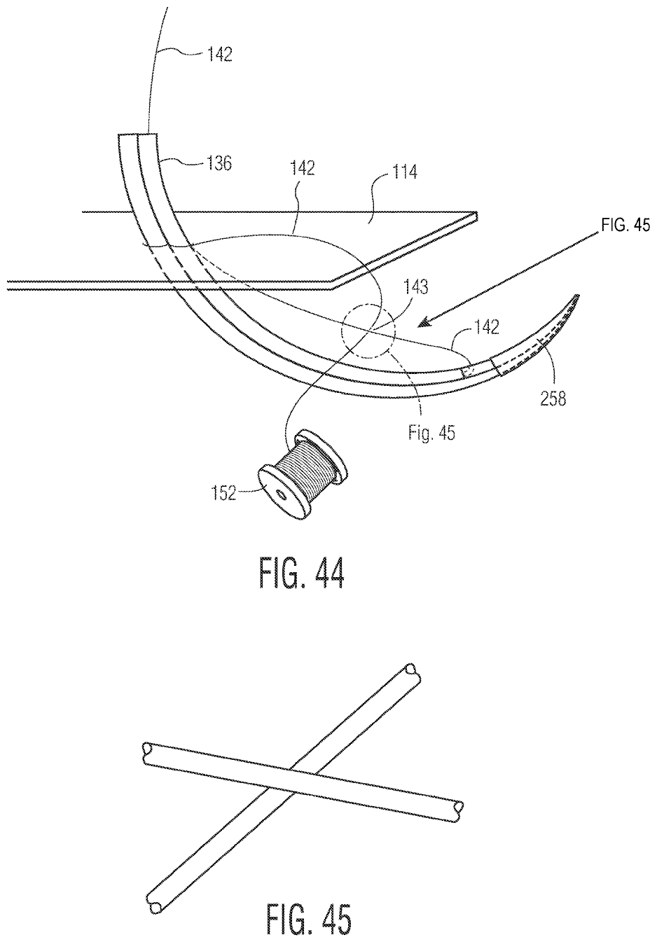

The main purpose of the penetration of the locking pin 234 in the channel 236 is to prevent the handle lever 108 from re-closing (Arrow B) and thus to prevent the curved needle assembly 136 from actuating and penetrating the tissue 114 (FIG. 43) a second time until after the user has rotated the first thumb lever 200 upwardly (dotted Arrow F of FIG. 11) , which drives the first drive axle 198 and respective mutilated spur gear 194 to engage the rack 190 and bobbin carrier 168 and impart linear motion, in the direction of Arrow E (FIGS. 4, 5, and 7), of the bobbin carrier 168 from the actuated position 176 (as illustrated in FIG. 5) to the rest position 174 (as illustrated in FIG. 4). An upward rotation (dotted Arrow F of FIG. 11) of the first thumb lever 200 moves the first thumb lever from the first thumb lever second position (as illustrated in FIGS. 10 and 12) to the first thumb lever first position (as illustrated in FIGS. 2, 7, 8, 9, and 11). In other words, the penetration of the locking pin 234 in the channel 236 prevents the handle lever 108 from accidentally or unintentionally re-closing (Arrow B)(which would cause the curved needle assembly 136 to re-penetrate the tissue 114 (FIG. 43) a second time) until after the user (e.g. the surgeon) has rotated the first thumb lever 200 upwardly (dotted Arrow F of FIG. 11) to move the bobbin 152 across the XY plane to reset and ready the handheld medical suturing device 100 for an intended, second penetration of the tissue 114, as exemplified in FIG. 53.

In such embodiment, the at least one locking pin of the handheld medical suturing device 100 is configured for withdrawal from the channel upon a rotation of the first thumb lever by a user from the first thumb lever second position (as illustrated in FIGS. 2, 7, 8, 9, and 11) to the first thumb lever first position.

The first thumb lever 200 is typically used by a right-handed user grasping the handheld medical suturing device 100. The second thumb lever 212 is typically used by a left-handed user grasping the handheld medical suturing device 100.

As noted above, referring to FIGS. 7-12, there is preferably a second thumb lever 212, which extends outwardly of the handle body 102, and the second thumb lever 212 is connected to the second drive axle 210 which has a second spur gear 208 that operatively engages the first spur gear 206, to impart rotational movement, in the direction of Arrow F in FIG. 7. A rotation of the second thumb lever 212 causes the second spur gear 208 to rotate the first spur gear 206. Accordingly, a downward rotation of the second thumb lever 212 (in the direction of solid Arrow Fin FIG. 11) causes the at least one locking pin 234 to align with the channel 236, as exemplified in FIG. 54, upon such downward rotation of the second thumb lever 212 from the second thumb lever first position, as illustrated in FIGS. 2, 7, 8, 9, and 11, to the second thumb lever second position, as illustrated in FIGS. 10 and 12. When the handle lever 108 is pivoted (re-opened) in the direction of Arrow C in FIGS. 1 and 55, the locking pin 234 penetrates and slides into the channel 236 of the handle body 108 into a locked position, as exemplified in FIG. 55.

Moreover, since the second spur gear 208 engages the first spur gear 206, then a rotation of the second thumb lever 212 causes the mutilated spur gear 194 to operatively engages the rack 190 to translate and impart rotational motion, in the direction of Arrow D, of the mutilated spur gear 194 into linear motion, in the direction of Arrow E, of the rack 190 from the rest position 174 to the actuated position 176, upon the rotation, in the direction of Arrow F, of the second thumb lever 212 from a second thumb lever first position (as illustrated in FIGS. 2, 7, 8, 9, and 11) to a second thumb lever second position (as illustrated in FIGS. 10 and 12). The linear motion (Arrow E) of the rack 190 moves the bobbin carrier 168 and a bobbin 152 side to side in the direction of Arrow E within the fixed bobbin channel 156.

As can be appreciated from this description, the mutilated spur gear 194 operatively engages the rack to translate its rotational motion into linear motion of the rack upon rotation of either the first thumb lever 206 or the second thumb lever 212. The linear motion of the rack 190 moves the bobbin carrier 168 and the bobbin 152 side to side within the bobbin channel.

In an embodiment, the handheld medical suturing device 100 further comprises at least one spool 148 of suture 142 that is disposed within an inner cavity passageway 150 that is defined by the proximal end 110 of the handle body 102. The suture 142 is preferably threaded from the at least one spool 148 through the inner cavity passageway 150 to the curved suturing needle assembly 136. The suture 142 is further threaded from the curved suturing needle assembly 136 to the bobbin 152 that is disposed within the distal end 111 of the handle body 102. The at least one spool 148 is adapted to dispense the suture 142 to the curved suturing needle assembly 136 upon the rotation of the curved suturing needle 136 by a user from the retracted position (as illustrated in FIGS. 1 and 15) to the fully extended position (as illustrated in FIGS. 3 and 17), upon the pivoting of the handle lever 108 (in the direction of Arrow Bin FIG. 18) relative to the handle body 102 from the open position (as illustrated in FIGS. 1, 2, and 15) to the squeezed position (as illustrated in FIGS. 3 and 17).

As illustrated in FIGS. 4-5, the bobbin 152 has a proximal end 214 opposite a distal end 216, and the proximal end 214 preferably includes a first plurality of annularly disposed ridges 218 that provide a first frictional resistance against rotation of the bobbin 152 within the bobbin carrier 168 in order to keep the suture 142 taut, as the curved suturing needle assembly 136 is actuated and penetrates through the tissue 114, between the retracted position and the fully extended position, upon the pivoting of the handle lever 108 (Arrow B) relative to the handle body 102. Such frictional resistance of the first plurality of annularly disposed ridges 218 also maintains tension 142 on the suture upon the movement of the bobbin 152 along the central axis across the XY plane between a rest position 174 and an actuated position 176.

In another embodiment, the distal end 216 of the bobbin 152 preferably includes a second plurality of annually disposed ridges 220 that provide a second frictional resistance against rotation of the bobbin 152 within and relative to the bobbin carrier 168, upon the rotation of the curved suturing needle assembly 136 through the tissue between the retracted position and the fully extended position, upon the pivoting of the handle lever (Arrow B) relative to the handle body 102. Such second frictional resistance is also maintains tension on the suture 152 upon a user's movement of the bobbin 152 along the central axis across the XY plane (FIG. 4) between the rest position 174 and the actuated position 176.

Referring to FIG. 4, in yet another embodiment, the handheld medical suturing device 100 has at least one magnet 222 disposed within a wall 224 of the bobbin carrier 168, in a spaced relation to the bobbin 152, and the bobbin 152 comprises a ferrous material 226 that provides a magnetic attraction which provides a magnetic resistance against rotation of the bobbin 152 relative to the bobbin carrier 168, in order to keep the suture taut. Such magnetic resistance imparts tension on the suture 142.

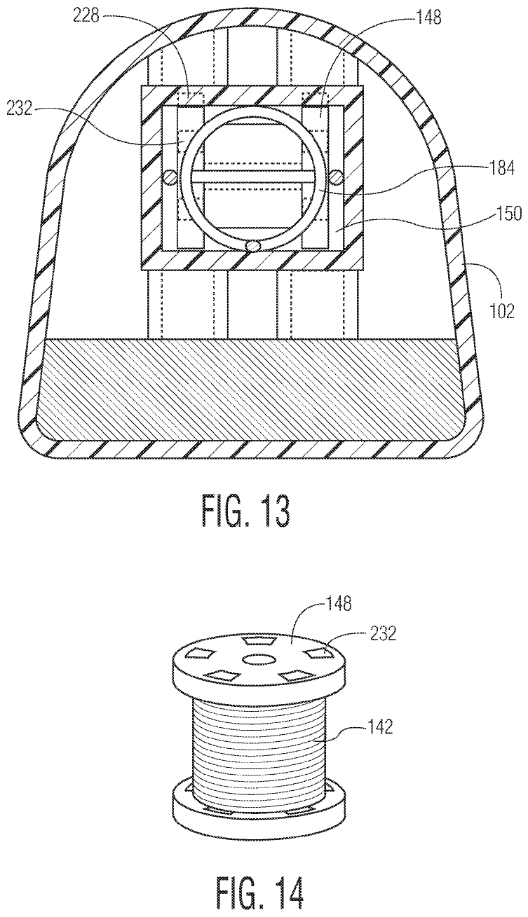

Referring to FIGS. 13-14, in another embodiment, the handheld medical suturing device 100 preferably includes at least one magnet 228 that is disposed within a wall 230 of the handle body 102, and the handheld medical suturing device 100 further comprises at least one spool 148 of suture that is disposed within the inner cavity passageway 150 that is defined by the proximal end 110 of the handle body 102. The at least one spool 148 preferably includes a ferrous material 232 that is adapted to provides a magnetic attraction to said at least one magnetic disc 228 which provides magnetic resistance against rotation of the at least one spool 148 relative to the wall of the handle body 102, in order to keep the suture 142 taut.

In another embodiment, the handheld medical suturing device includes at least one frictional compression disc disposed in the handle body between the at least one spool 148 and an adjacent wall of the handle body. The at least one frictional compression disc provides frictional resistance against rotation of the at least one spool 148 to provide tension on the suture 142, to help keep the suture 142 taut.

In an embodiment, the handheld medical suturing device 100 includes at least one spool 148 of suture 142 disposed within the inner cavity passageway 150 defined by the proximal end 110 of the handle body 102, where suture 142 is threaded from that at least one spool 148 through the inner cavity passageway 150 to the curved suturing needle assembly 136, and the suture 142 is further threaded from the curved suturing needle assembly 136 to the bobbin 152 that is disposed within the distal end 110 of the handle body 102, as illustrated in FIG. 2. The at least one spool 148 is adapted to dispense the suture 142 to the curved suturing needle assembly 136 upon the rotation (Arrow A) of the curved suturing needle assembly 136 from the retracted position (as illustrated in FIGS. 1 and 15) to the fully extended position (as illustrated in FIGS. 3 and 17).

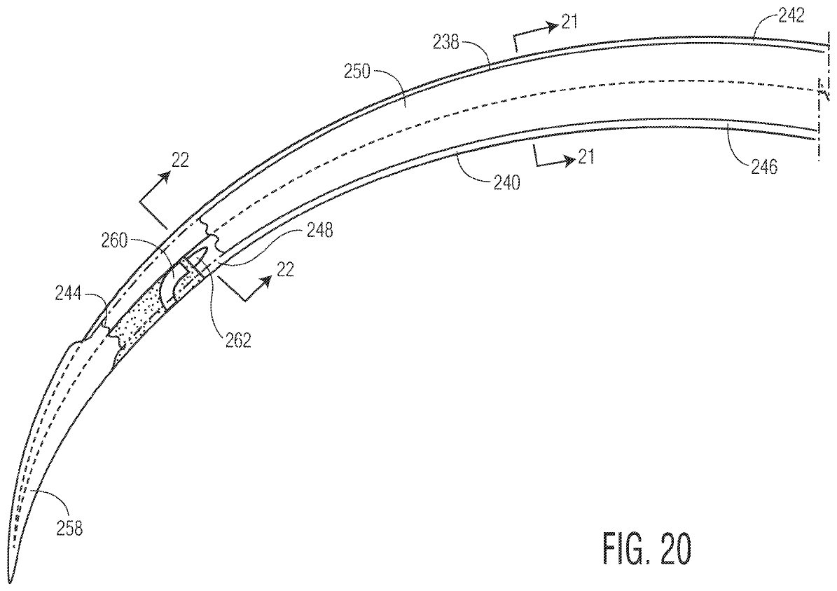

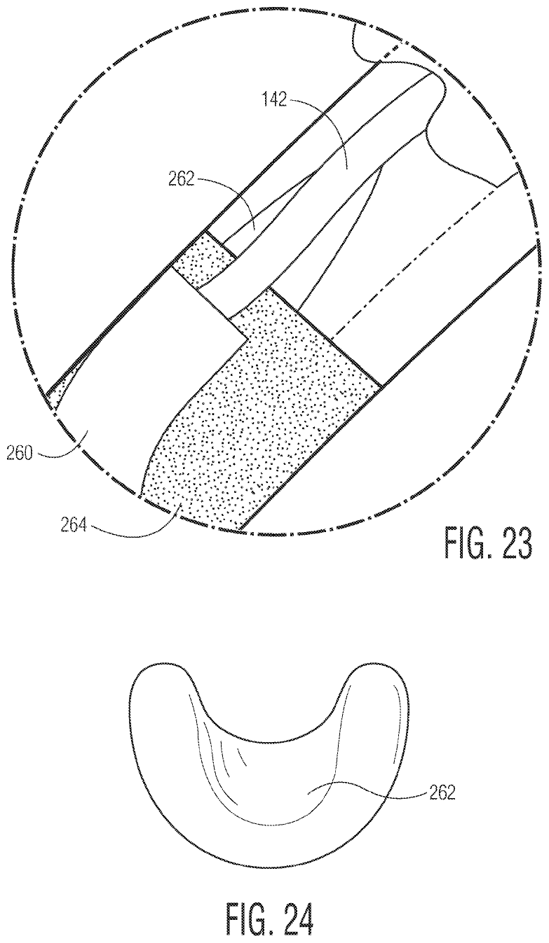

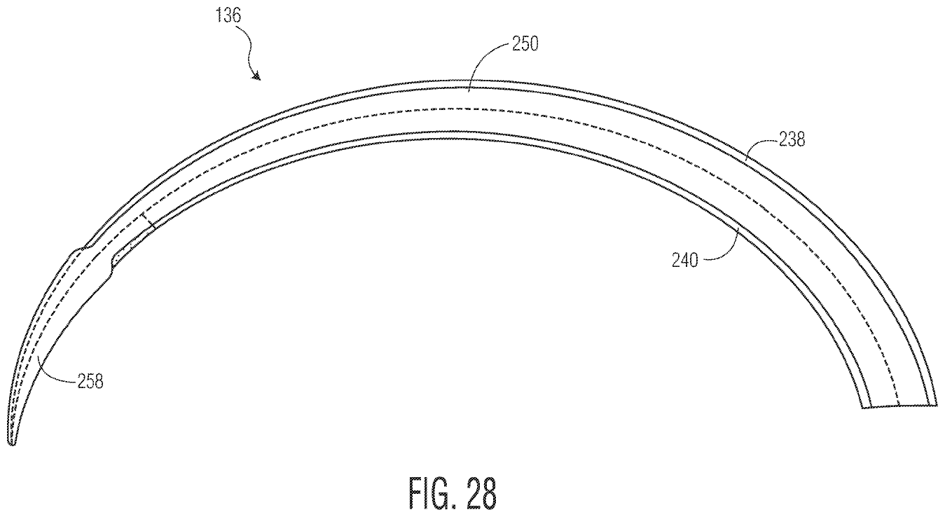

Referring to FIGS. 20-28, in an embodiment, the curved suturing needle assembly 136 of the handheld medical suturing device 100 comprises an outer curved needle 238 fixed to an inner curved needle 240. The outer curved needle 238 has a proximal end 242 opposite a distal end 244. The inner curved needle 240 has a proximal end 246 opposite a distal end 248. The curved suturing needle assembly 136 preferably includes a shroud 250 that is disposed along an outer surface 252, 254 of the outer curved needle 238 and of the inner curved needle 240, as illustrated in FIGS. 20-22 and 28. The shroud forms a passage 256 for the suture 142 to thread therethrough.

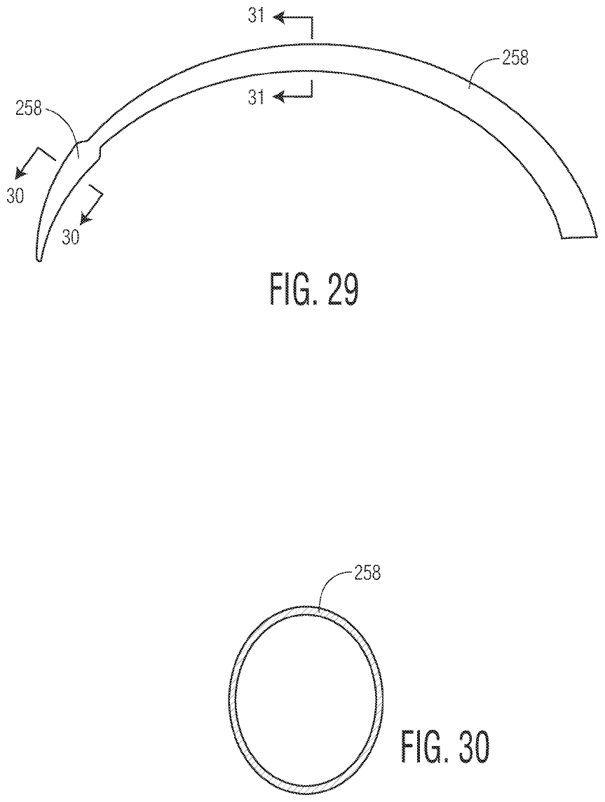

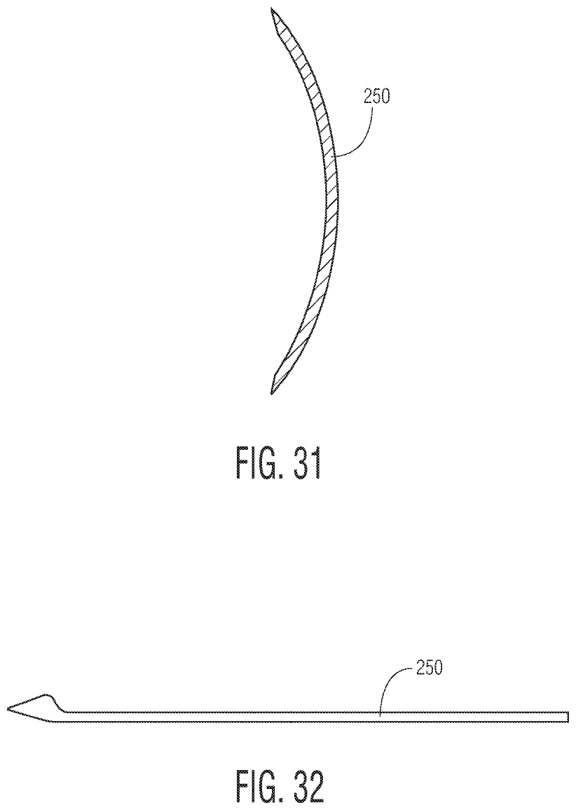

The shroud 250 includes a pointed cover 258 encapsulating the distal end 244 of the outer curved needle 238, as seen in FIGS. 20, 25, and 27-30. Apart from the pointed cover 258 encapsulating the distal end 244 of the outer curved needle 238, the shroud primarily comprises a body having a curved cross-section, as illustrated in FIGS. 31-32.

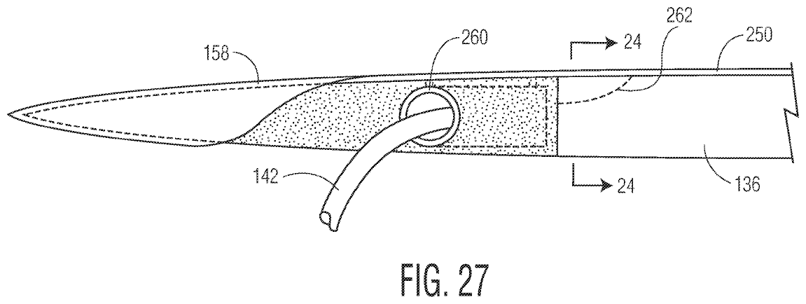

In yet another embodiment, the handheld medical suturing device 100 includes an elbow guide 260 disposed within the pointed cover 258, as illustrated in FIGS. 20, 23, and 26 27. The elbow guide 260 engages and guides the suture 142 as the suture 142 extends outwardly from and exits the distal end 248 of the inner curved needle 240. Preferably, a filling 264 is provided within the pointed cover 258 of the shroud 250, around the elbow guide 260. As illustrated in FIGS. 22-24, the distal end 248 of the inner curved needle 240 preferably defines a ramp 262 to engagingly guide the suture 142, so as to provide a smooth exit for the suture 142 as it threads out of the inner curved needle 240, as illustrated in FIG. 27.

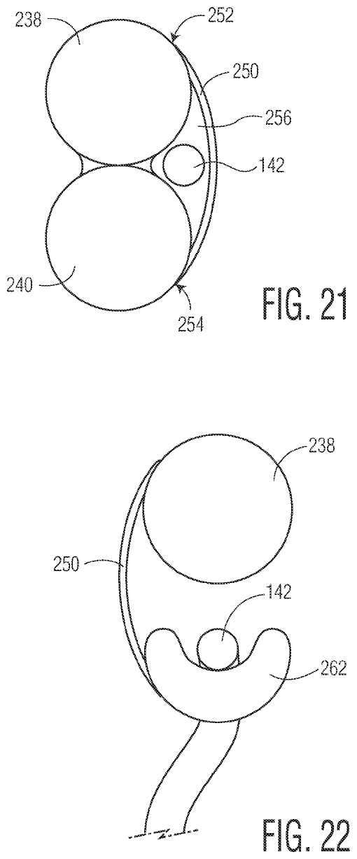

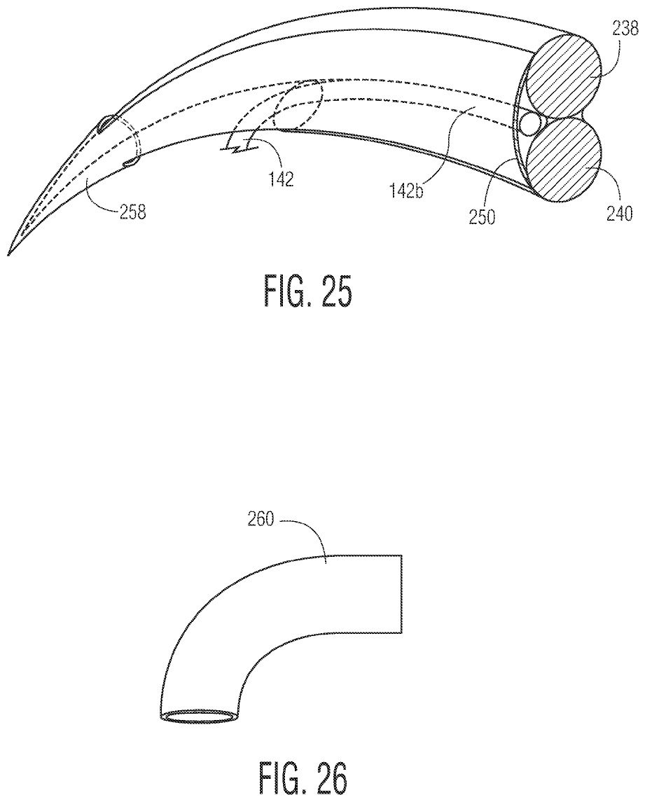

In an embodiment, the handheld medical suturing device 100 includes a single length of suture 142, where a first portion 142a of the single length of suture is disposed around the at least one spool (as illustrated in FIG. 14), where a second portion 142b of the single length of suture is disposed along the curved suturing needle assembly 136 (FIGS. 21 and 25), where a third portion 142c of the single length of suture is disposed inside the bobbin carrier cavity (FIG. 1), and where a fourth portion 142d of the single length of suture is disposed around the bobbin 152 (FIG. 4). The single length of suture 142 is also exemplified in FIGS. 40-41.

In another one embodiment, the single length of suture 142 includes a first portion 142a of the single length of suture disposed around the at least one spool 148 (FIG. 14), a second portion 142b of the single length of suture 142 is disposed within the shroud 250 (FIGS. 21 and 25) of the curved suturing needle assembly 136, a third portion 142 c of the single length of suture 142 is disposed inside the bobbin carrier cavity 172 (FIG. 4), and a fourth portion 142d of the single length of suture 142 is disposed around the bobbin 152 (FIG. 4).

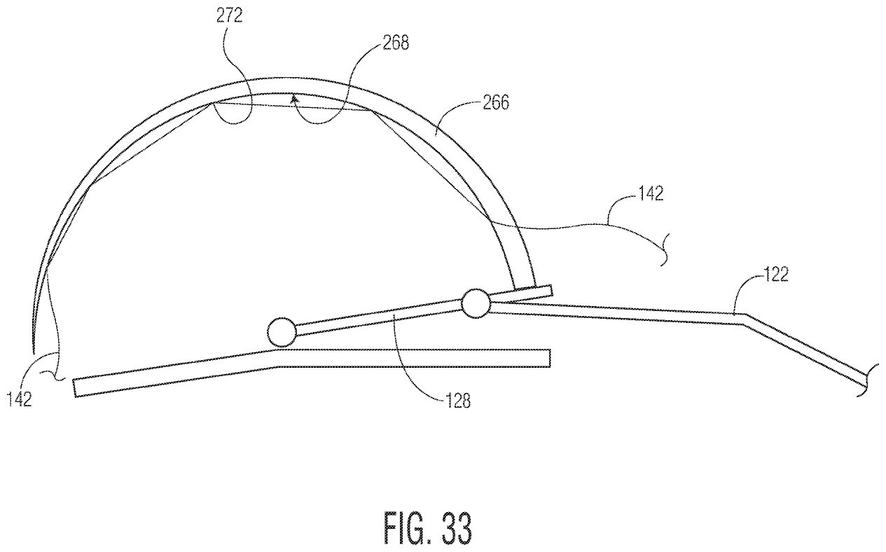

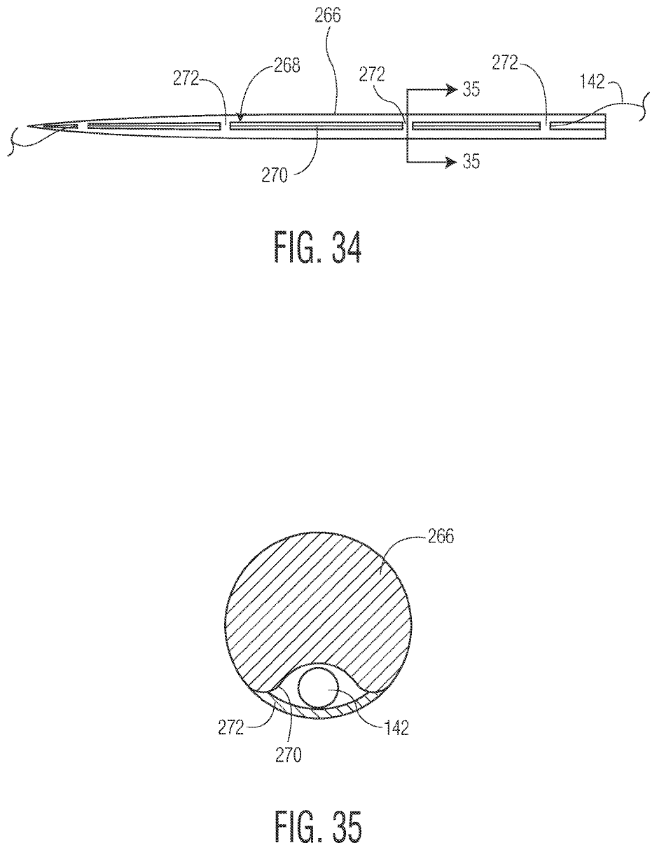

Referring to FIGS. 33-35, in another embodiment, the curved suturing needle assembly 136 comprises a single curved needle 266 having an inner surface 268 which defines an annular groove 270. The single curved needle 266 preferably has a plurality of containment bridges 272 annularly disposed along the annular groove 270 of the inner surface 268. The plurality of containment bridges 272 maintain the suture 242 within the annular groove 270 of the single curved needle 266, as illustrated in FIGS. 33 and 35.

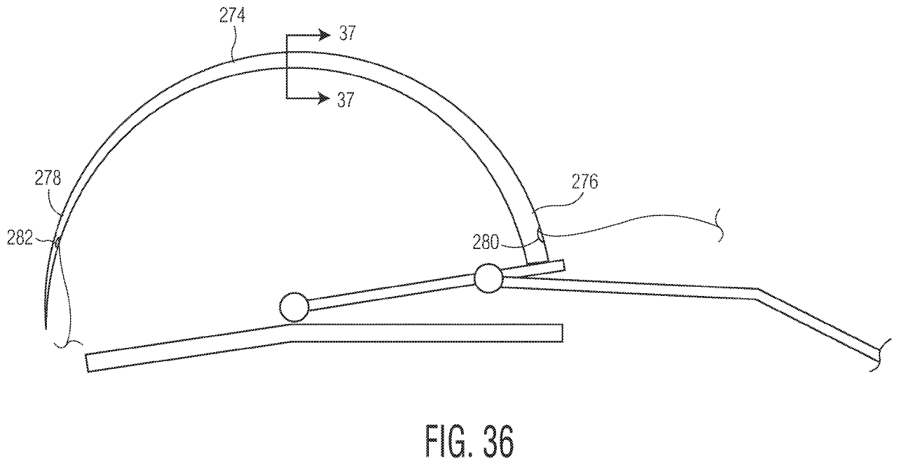

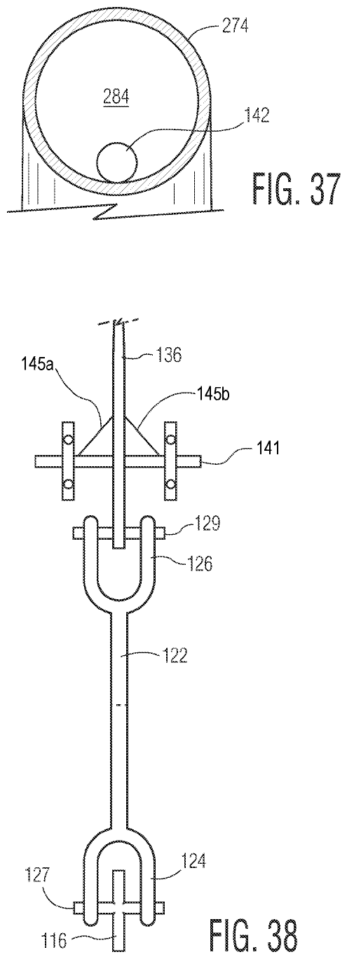

Referring to FIGS. 36-37, in another embodiment, the curved suturing needle assembly 136 includes a single curved needle that is a hollow needle, and suture is threaded through the hollow needle. In such embodiment, the curved suturing needle assembly 136 includes a curved hollow needle body 274 having a proximal end 276 opposite a (pointed) distal end 278. The proximal end 276 of the curved hollow needle body defines a first orifice 280, for suture 142 to enter, as illustrated in FIG. 36. The distal end 278 of the curved hollow needle body 274 defines a second orifice 282, for the exit of the suture 142. As seen in FIG. 27, the curved hollow needle body 274 defines a passage 284 therethrough between the first orifice 280 and the second orifice 282, and the suture 142 is threaded through the first orifice 280, through the passage 284, and through the second orifice 282.

As further embodied and described herein, there is provided a curved suturing needle assembly 136 having an outer curved needle 238 fixed to an inner curved needle 240. In one embodiment, the outer curved needle 238 is soldered to the inner curved needle 240, as illustrated in FIG. 21. In another embodiment, the outer curved needle 238 and the inner curved needle 240 are jointly fabricated by being cast in a single mold.

The outer curved needle 238 needle has a proximal end 242 opposite a distal end 244. The inner curved needle 240 has a proximal end 246 opposite a distal end 248. In an embodiment, the disclosed curved suturing needle assembly 136 includes a shroud 250 disposed along an outer respective surface 252, 254 of the outer curved needle 238 and of the inner curved needle 240. The shroud forms a passage 256 for the suture 142. Preferably, the shroud 250 comprises a pointed cover 258 encapsulating the distal end 244 of the outer curved needle 238.

In yet another embodiment, the disclosed curved suturing needle assembly 136 includes an elbow guide 260 disposed within the pointed cover 258 of the shroud 250. The elbow guide 260 is configured to engage and guide the suture 142 when the suture 142 extends outward from the distal end 248 of the inner curved needle. A filling 264 is preferably disposed within the pointed cover 258 of the shroud 250. The distal end 248 of the inner curved needle 240 preferably defines a ramp 262 adapted to engagingly guide the suture 142, to provide for a smooth exit for the suture 142 exiting the distal end 248 of the inner curved needle 240 of the curved suturing needle assembly 136.

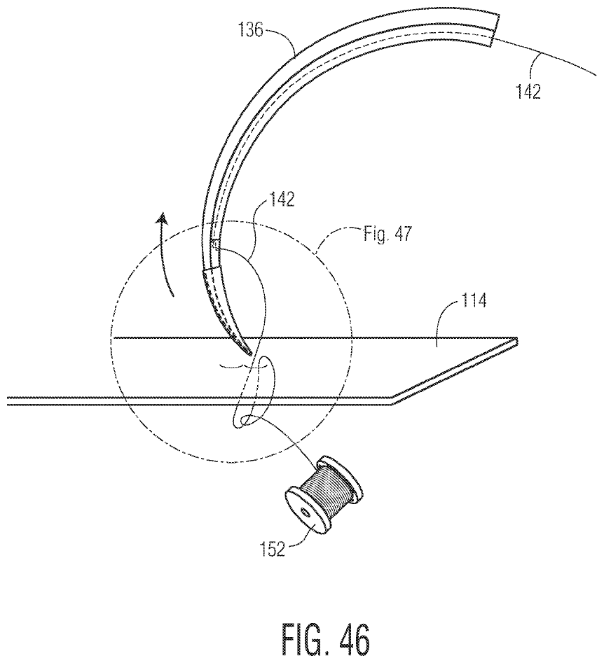

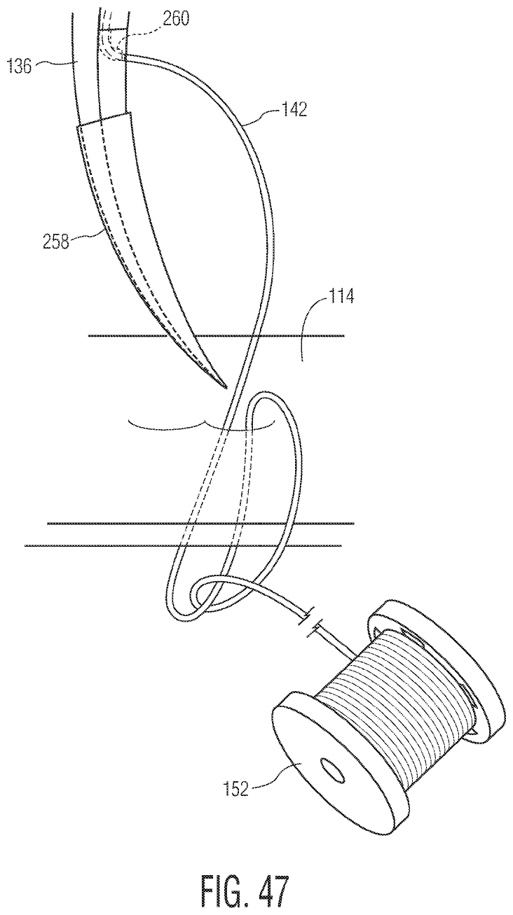

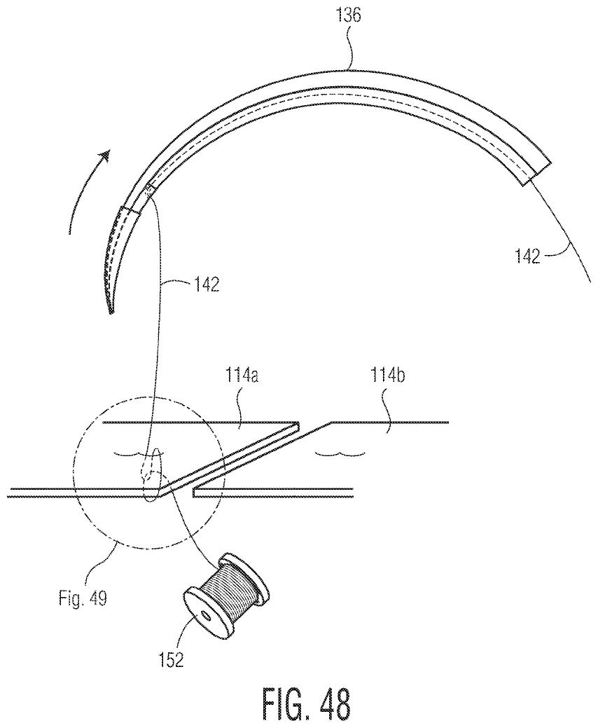

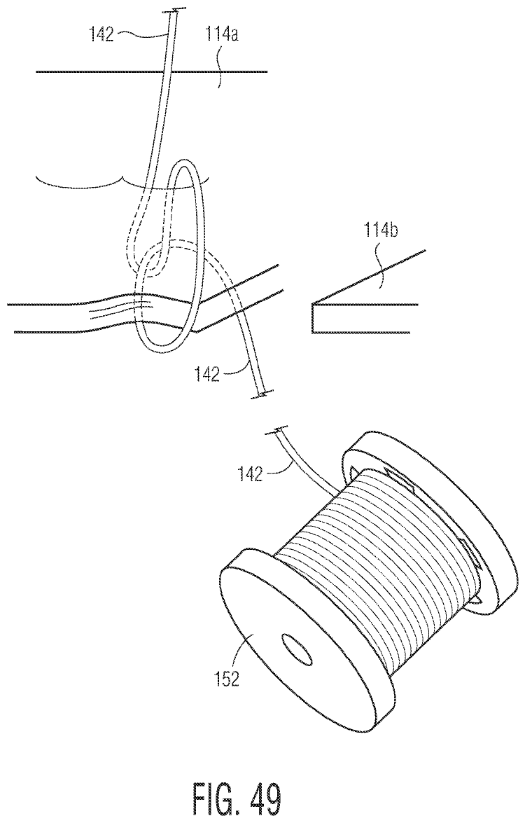

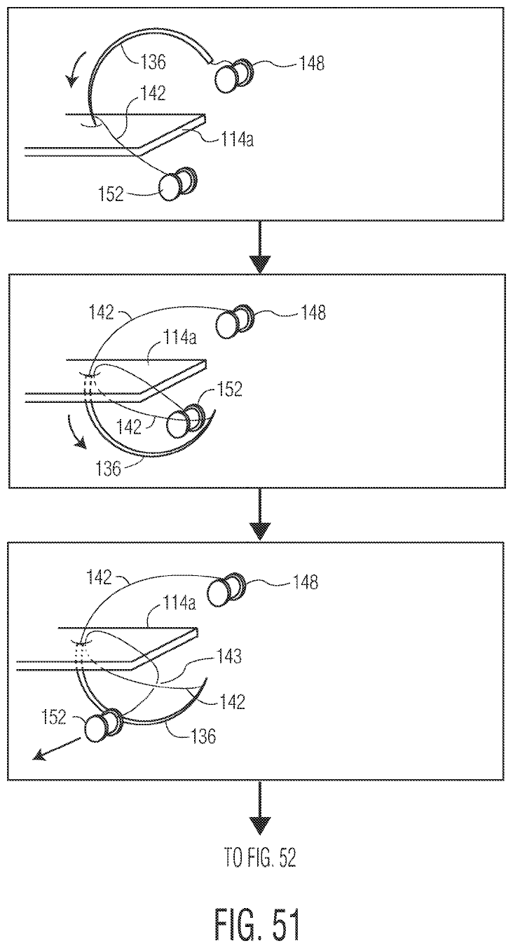

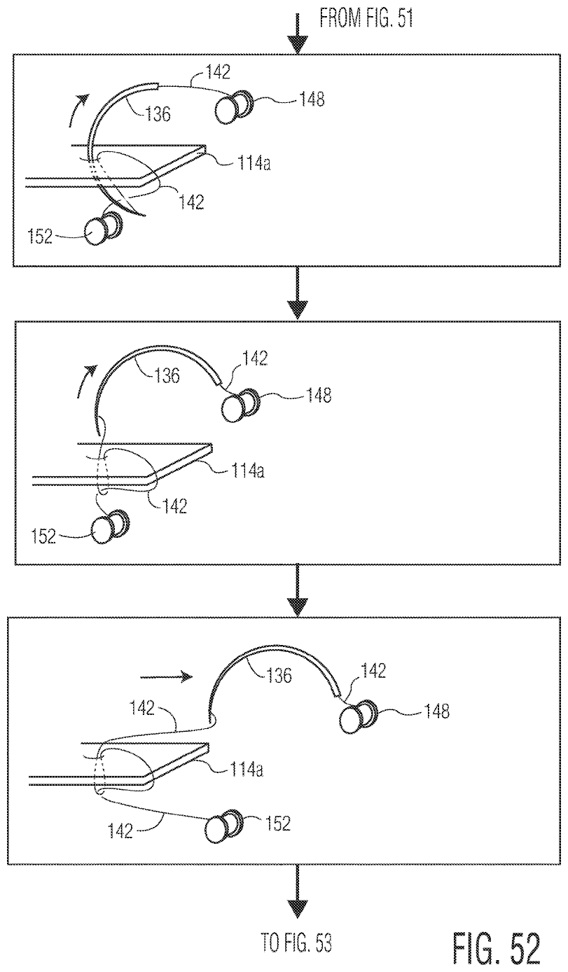

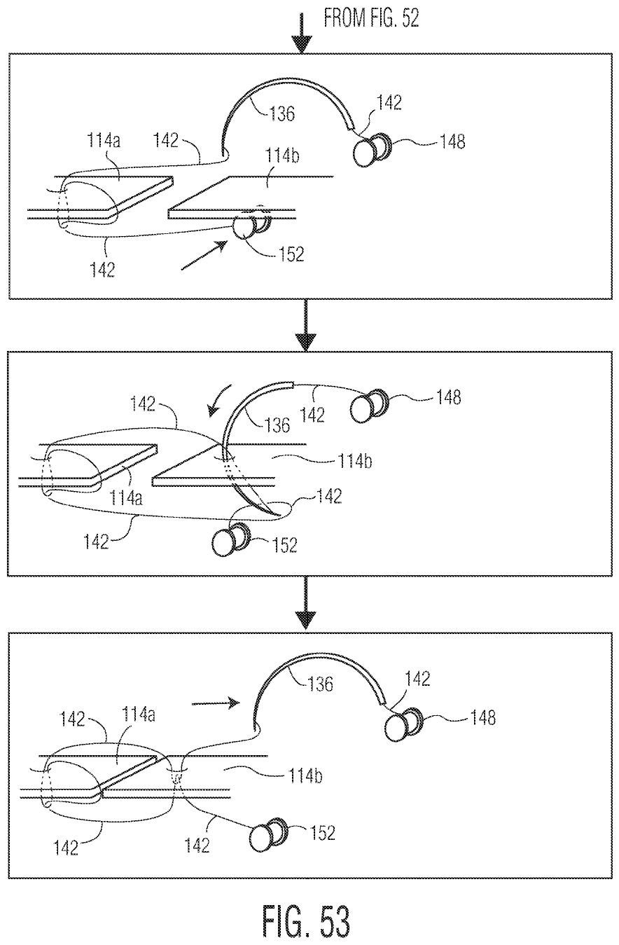

FIGS. 41-55 illustrate the path of the suture and bobbin as the bobbin 152 moves side to side in the fixed bobbin channel 156. FIG. 51 illustrates the bobbin 152 forming a bow by crossing the XY plane and further illustrates the tying of a knot around the tissue 114. FIG. 52 illustrates the curved suturing needle assembly 136 of the handheld medical suturing device 100 completing the first knot around the first tissue 114a and moving to a second tissue 114b, as illustrated in FIG. 53.

As illustrated in FIG. 53, embodiments of the handheld medical suturing device 100 of the disclosed invention locks the suture 142 at each interval, thereby providing an efficient manner of implementing continuous interlocking sutures, which lock at each interval, which provides for a stronger suture or stich between adjoining tissues (e.g., 114a and 114b) , overcoming the problems associated with conventional running or continuous suturing techniques.

One or ordinary skill in the art can appreciate from this disclosure that embodiments of the disclosed invention can be fabricated at a small size such that such embodiments can also be used endoscopically and/or laparoscopically.

Non-robotic minimally invasive surgery is also known as endoscopic surgery, which is a minimally invasive procedure that utilizes an endoscope to reach internal organs through very small incisions. In other words, embodiments of the present disclosure can be used for endoscopic suturing or stitching through an access tube or the like, preferably at the end of an elongated shaft.

Laparoscopic surgery involves making several small incisions in the abdomen. Gas is then used to distend the abdomen, to create space for the operation. A surgeon uses a laparoscope, which has a video camera attached, to examine the area with visual references on a monitor. Specialized keyhole instruments are inserted to perform the operation. Embodiments of the present disclosure can be used for laparoscopic suturing or stitching with a laparoscope.

It is well known that a suture closure of the abdominal wound typically takes place at the end of a surgical procedure, and such procedure often takes several hours, depleting the surgeon of energy, which often causes the surgeon to be less precise with the closure technique utilized by the surgeon. As previously noted above, when smaller sutures (6 mm to 8 mm from the fascial wound edge) are placed more closely together to yield a suture-to-wound length ratio of 4:1 or greater, that cuts the incidence of incisional hernias by half. One reason why the recommendations for closely placed sutures are rarely followed is because that requires a substantial increase in the number of suture points. In other words, the recommended modifications of smaller, more numerous, and more closely placed suture points are rarely followed when a surgeon utilizes conventional suturing devices, because they demand more effort and time at a moment at the end of the surgery when the surgeon is tired from several hours of surgery.

As can been seen from the summary, description of the drawing figures, detailed description, and the figures herein, embodiments of the invention greatly facilitate the performance of the recommended improved technique of placing more closely spaced sutures by making the suturing process mostly automatic, as described in detail above, as the disclosed invention makes suturing much less labor intensive and makes the process much faster than conventional techniques, and thus the embodiments disclosed herein make the recommendations more likely to be adhered to.

Referring to FIGS. 1-3 and 19, the handheld medical suturing device 100 has a short opening 112 for a tissue bite 113, which as illustrated in FIG. 19, is preferably 6-8 mm between the back of the opening 112 of the handle body 102 and the puncture point 119, (where the curved needle suturing assembly 136 will penetrate the tissue). Such leads to the sutures (114, 114a, 114b) being automatically placed approximately 6 mm to 8 mm inside the wound edge, as is a preferred technique, as exemplified in FIG. 19, where the tissue bite distance 113 is preferably 6-8 mm.

The handheld medical suturing device 100 also preferably includes a simple visual aid 115 (preferably in the form of a line 115 in the opening 112) to measure the distance 117 between adjacent suture points, and such distance 117 preferably aligns with the centerline of the opening 112, as measured from the sidewall of the handle body 102, as illustrated in FIG. 19. The preferred distance 117 is 6-8 mm, and the distance 117 is most preferably 7 mm. In another embodiment, additional measurements points (such as those inscribed or illustrated on a tape measure) are inscribed or illustrated within the opening 112 of the handheld medical suturing device 100 to assist the surgeon in measuring the distance between suture points. Thus, the handheld medical suturing device 100 better facilitates compliance with the recommendation for closeness between suture points in the tissue.

As can be appreciated from the disclosure herein, the handheld medical suturing device 100 provides a means to achieve the goal of preventing many incisional hernias by making the suturing process compliant with the recommendations in a faster, easier, more precise, and more repeatable manner, which is much more likely to be performed by the surgeon in light of the ease of use and semi-automatic nature of the handheld medical suturing device 100.

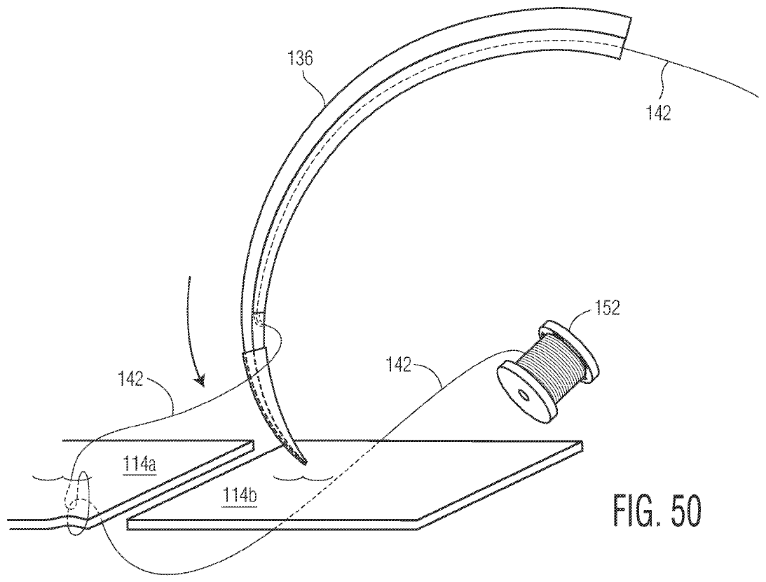

The handheld medical suturing device 100 uniquely adds stability to the wound (114a, 114b) by running an outer length of suture (one suture outside the wound) and contemporaneously running an inner length of suture (a suture inside the wound) simultaneously, as illustrated in FIGS. 50-53, along parallel axes. As such, it can be appreciated that the disclosed handheld medical suturing device 100 uniquely distributes the tension between these outer and inner lengths of suture, rather than just over a single suture as done conventionally.

A person's use of the handheld medical suturing device 100 is much more efficient than the current conventional method of continuous suturing, which requires numerous steps for each suture point, including, at least: (1) presentation of the needle, (2) clasping of the needle by the driver in the dominant hand, (3) stabilizing of the tissue by the non-dominant hand with a forceps, (4) perforation of the tissue by the clasped needle, (5) releasing the clasp on the needle, (6) rotating the user's hand to prepare for a re-clasping of the needle, (7) re-clasping the needle on the opposite surface of the tissue, (8) withdrawing the needle from such opposite surface of the tissue, and (9) repositioning the clasp on the needle, and (10) re-clasping the needle with the driver in preparation for perforation of the tissue again.

As disclosed in the embodiments herein, the handheld medical suturing device 100 requires provides for better and more efficient suturing, requiring less steps than conventional device methods, including: (1) stabilizing of the tissue 114 with a surgeon's non-dominant hand with a forceps, (2) squeezing the handle lever 108 of the handheld medical suturing device 100 to perforate the tissue 114, and (3) rotating the first thumb lever 200 (in the direction of Arrow Fin FIG. 11) from the lever first position, as illustrated in FIGS. 2, 7, 8, 9, and 11, to the first thumb lever second position, as illustrated in FIGS. 10 and 12, and (4) releasing the handle lever 108 to withdraw the needle from the tissue 114 to lock the suture onto itself (i.e., tie a knot), as exemplified in FIGS. 44-52, and thereby form a locked suture. As can be appreciated from this disclosure, the handheld medical suturing device 100 is comparatively simple in its construction and arrangement, strong, durable, efficient in its use, readily set up in operative position with respect to tissue or fabrics, and comparatively inexpensive to manufacture. The handheld medical suturing device 100 is single use, meaning, it is preferably disposable of after being utilized to suture the tissue of a single patient.

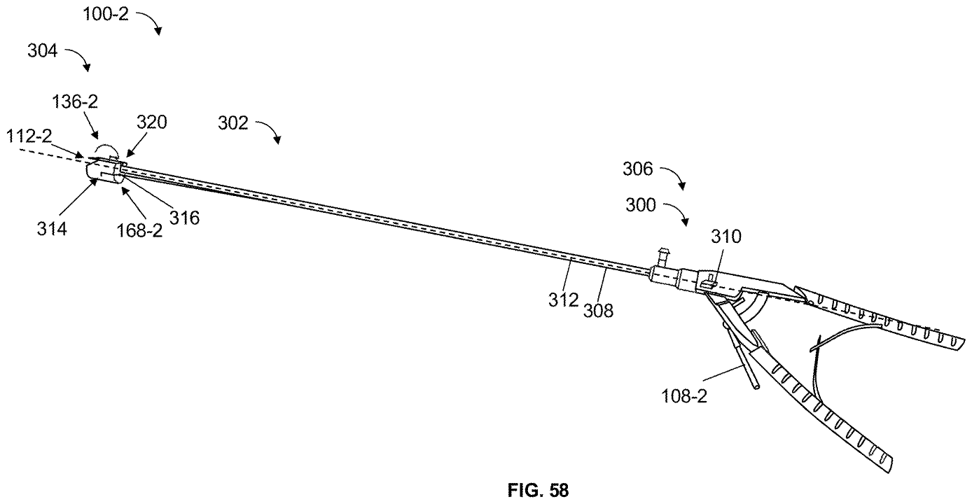

The next section describes another medical suturing device 100-2 with reference to FIGS. 58-67, according to various examples. For example, FIG. 58 illustrates a medical suturing device 100-2, according to at least one example. The medical suturing device 100-2 may be incorporated into a laparoscopic device 302 having an elongated body 308 with a distal end 304 and a proximal end 306. The suturing device 100-2 may be sized and shaped for use in minimally invasive procedures, such as laparoscopic, arthroscopic, and robot-assisted procedures, where access to tissue may be obtained through a cannula, trocar, port, or other narrow access pathway.

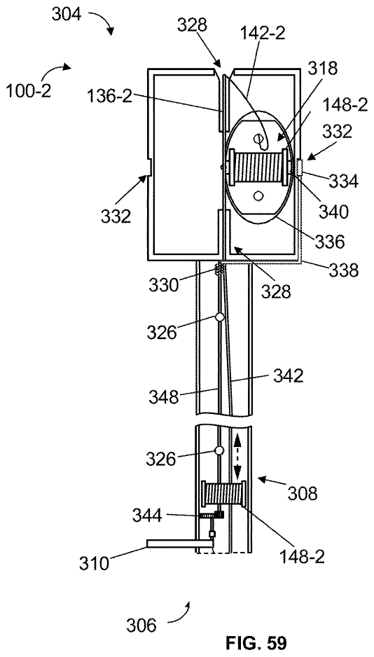

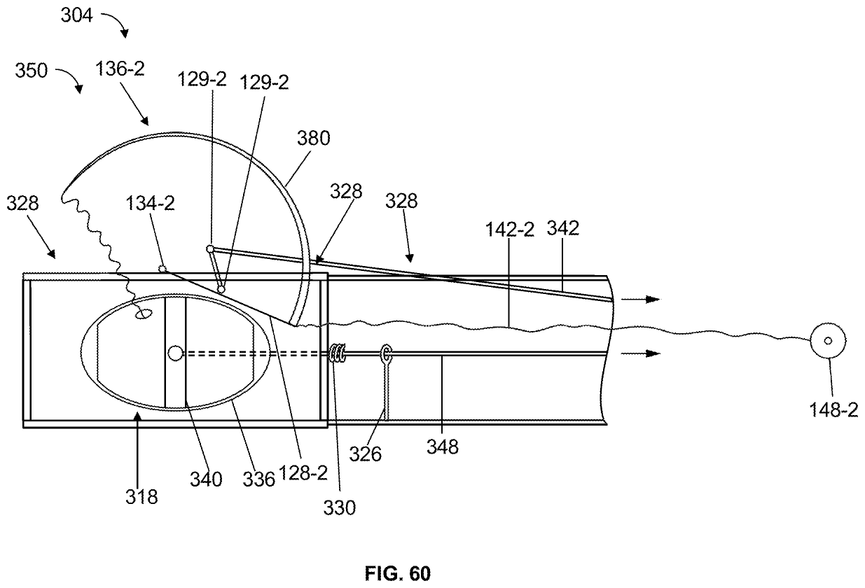

Referring now to FIG. 58, which illustrates a perspective view of medical suturing device 100-2, and to FIG. 59, which illustrates a cross-sectional side view of the suturing device 100-2, in accordance with embodiments of the invention. In various embodiments, the suturing device 100-2 may be configured as part of, mounted to, or otherwise incorporated into a laparoscopic device 302 having an elongated body 308 extending between a distal end 304 and a proximal end 306. The elongated body 308 may define a generally longitudinal axis 312 extending between the proximal end 306 and the distal end 304, and may be sized and shaped for introduction through a trocar, cannula, port, or other minimally invasive access structure.

In various embodiments, the elongated body 308 may include a shaft, tube, housing, or other structural body configured to support and/or protect components of the suturing device 100-2. For example, the elongated body 308 may enclose and support one or more motion-transmission components extending between the proximal end 306 and the distal end 304, such as one or more actuating arms, rods, shafts, linkages, cables, drive members, or combinations thereof. In this way, a control input applied at the proximal end 306 may be transmitted to one or more suturing components positioned proximal or at the distal end 304 while maintaining a relatively slender profile suitable for minimally invasive procedures, such as but not limited to laparoscopy, arthroscopy, and the like.

In various embodiments, the proximal end 306 may include or be operably coupled with a control assembly 300 configured to actuate the suturing device 100-2. For example, as illustrated in FIG. 58, a handle with one or more levers (e.g., thumb lever 310 and handle lever 108-2) may be arranged at the proximal end 306 of the elongated body 308. In various embodiments, the control assembly 300 may be manually operable by a clinician, may be driven by one or more drive mechanisms, or may be coupled with an instrument interface, such as a robotic interface, powered interface, or other machine-driven control system (not shown). In various embodiments, the control assembly 300 may be configured to cause movement of a curved needle assembly 136-2 positioned at the distal end 304, and may additionally or alternatively be configured to cause movement of a bobbin 318 or bobbin-related component (e.g., bobbin capsule 336) positioned at the distal end 304.

In various embodiments, the elongated body 308 may include one or more supports 326 positioned within the interior thereof. The supports 326 may be configured to maintain alignment of and/or support one or more components of the suturing device 100-2, to provide structural rigidity, and/or to guide movement of one or more movable elements extending between the proximal end 306 and the distal end 304. For example, the supports 326 may support one or more actuators, linkages, shafts, gear sets, springs, suture-handling components, or combinations thereof, such as motion transmitting components 342 and 348. In various embodiments, the supports 326 may assist in maintaining the relative positioning of components during actuation so that motion applied at the proximal end 306 is transmitted in a controlled manner to the distal end 304.

In various embodiments, one or more motion transmitting components may be positioned within the elongated body 308 and may be operably coupled with the control assembly 300. For example, a motion transmitting component 348 may be coupled with the thumb lever 310 such that movement of the thumb lever 310 causes corresponding movement of the component 348. In various embodiments, the component 348 may be configured to transmit motion toward the distal end 304 for purposes of moving the movable arm 338, the bobbin 318, the bobbin capsule 336, and/or another suturing component. In various examples, the component 348 may be supported by one or more supports 326 within the elongated body 308. In various embodiments, the component 348 and/or component 342 may include or define a rod, shaft, linkage, cable, wire, flexible shaft, push-pull member, threaded member, slider, rack, gear-driven member, or other drive member.

In various embodiments, one or more gear sets 344 may be included in the suturing device 100-2. The one or more gear sets 344 may be configured to translate, redirect, amplify, reduce, and/or otherwise modify motion generated at the proximal end 306. For example, the one or more gear sets 344 may convert rotational or pivoting movement associated with actuation of the thumb lever 310 into substantially linear motion of the component 348. In various embodiments, the one or more gear sets 344 may additionally or alternatively be configured to alter force transmission, adjust stroke characteristics, or facilitate packaging of the actuation components within the elongated body 308.

In various embodiments, a spool 148-2 configured to provide suture 142-2 for advancement by the curved needle assembly 136-2 may be positioned within the suturing device 100-2. For example, as shown in FIG. 59, the spool 148-2 may be positioned at or near the proximal end 306 of the elongated body 308. However, the spool 148-2 may be positioned at any suitable location within or coupled to the device 100-2 to provide suture 142-2 for use with the curved needle assembly 136-2. In various embodiments, positioning the spool 148-2 proximally may reduce crowding of components at the distal end 304 and may permit a more compact stitch-forming assembly, while other spool locations may be used depending on routing, packaging, or procedural considerations.

In various embodiments, one or more springs 330 may be included in the suturing device 100-2. The one or more springs 330 may be configured to bias one or more components toward a default position, to store and release energy during actuation, to assist in returning one or more components following actuation, and/or to maintain tension or positional stability within the device 100-2. For example, the one or more springs 330 may be operably associated with the thumb lever 310, component 348, movable arm 338, bobbin 318, bobbin capsule 336, or other components of the suturing device 100-2.

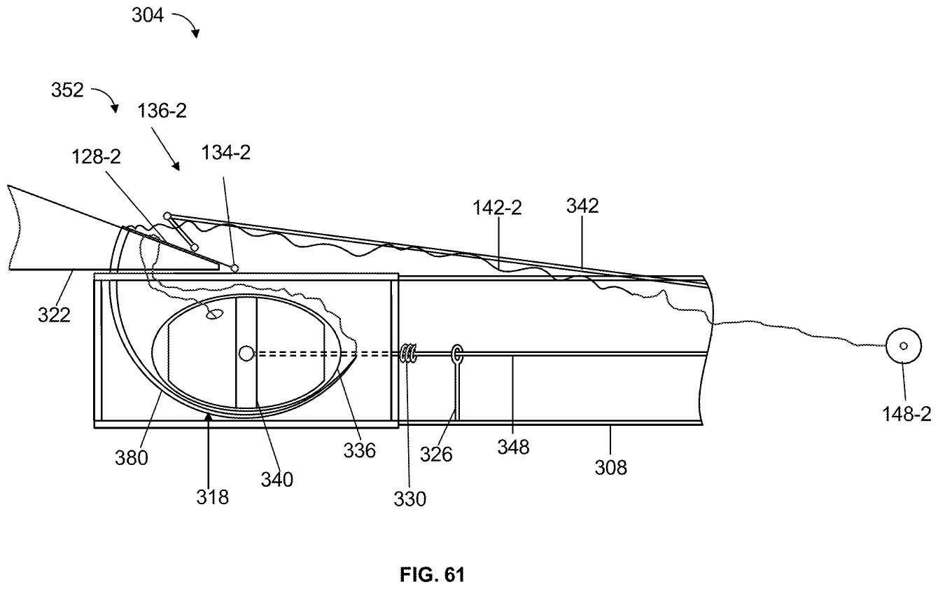

In various embodiments, the distal end 304 may support a stitch-forming or suturing assembly (e.g., curved needle assembly 136-2, bobbin carrier 168-2, and/or other components) adapted for operation within confined anatomical spaces. For example, the distal end 304 may include a curved needle assembly 136-2 configured to advance a first portion of a suture 142-2 through tissue 322 (as shown in and discussed in more detail with respect to FIGS. 60-61) and may further include a bobbin carrier 168-2 and a bobbin 318 configured to carry a second portion of the suture 142-2. In various embodiments, the bobbin carrier 168-2 may be arranged generally in-line with the curved needle assembly 136-2 along the longitudinal axis 312 of the elongated body 308, thereby providing a relatively narrow distal profile suitable for laparoscopic, arthroscopic, and other minimally invasive applications.

In various embodiments, the bobbin carrier 168-2 may be fixed relative to the elongated body 308 while the bobbin 318 moves relative to the bobbin carrier 168-2. Such an arrangement may reduce lateral space requirements at the distal end 304 of the elongated body 308 as compared with configurations in which a larger bobbin carrier structure moves transversely relative to the elongated body 308. This configuration may facilitate packaging of the stitch-forming components within a smaller distal footprint, may improve suitability for narrow-port access procedures, and may permit the locking-stitch functionality described herein to be implemented in laparoscopic, arthroscopic, and robot-assisted formats. In various embodiments, the bobbin 318 may be retained within a bobbin capsule 336, and the bobbin capsule 336 may move with or otherwise support the bobbin 318 within the bobbin carrier 168-2.

In various embodiments, the bobbin carrier 168-2 may include one or more slots 328. The one or more slots 328 may be configured to permit passage of at least a portion of the curved needle assembly 136-2 as the curved needle assembly 136-2 is actuated between retracted 350 and extended positions 352, such as shown in FIGS. 60 and 61. In various embodiments, the one or more slots 328 may accommodate movement of the curved needle assembly 136-2 while maintaining the compact arrangement of the bobbin carrier 168-2, bobbin 318, and/or bobbin capsule 336.

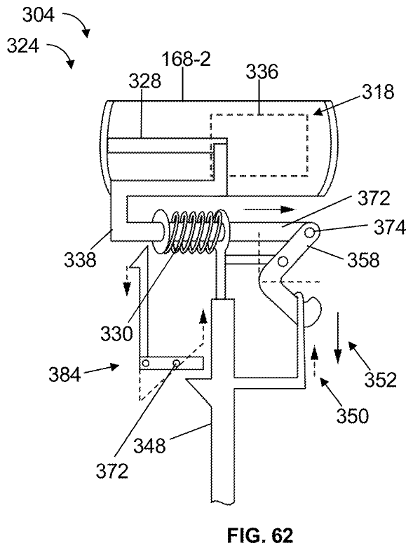

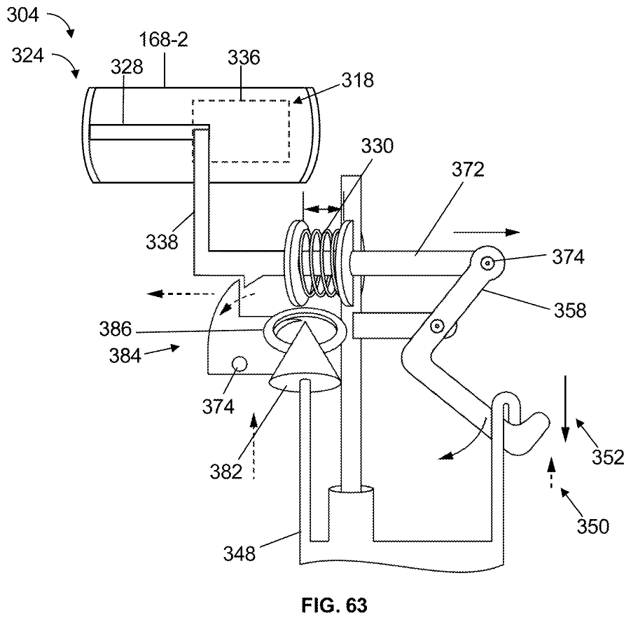

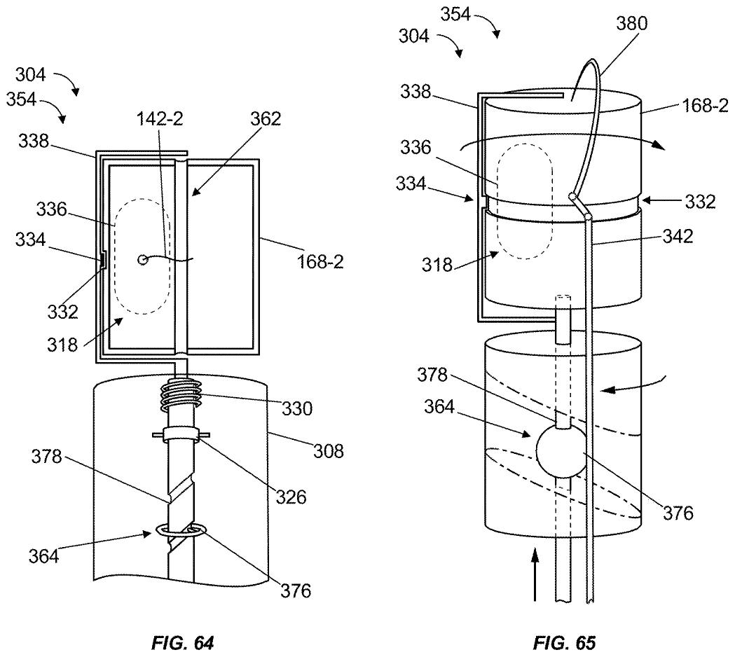

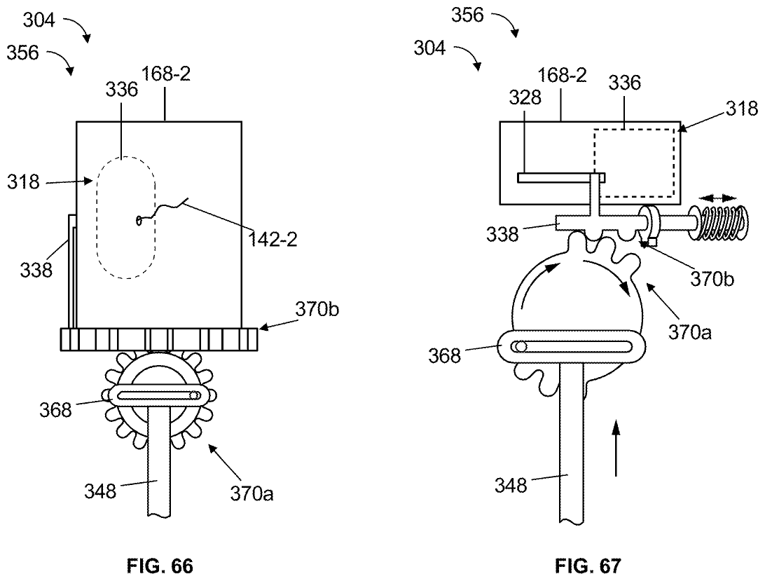

In various embodiments, movement of the bobbin 318 may be produced by a movable arm 338 and magnetic coupling arrangement. In various embodiments, movement of the movable arm 338 may be facilitated by a bell crank mechanism (such as shown in FIGS. 62 and 63), a linear actuator (such as shown in FIGS. 64 and 65), a scotch yoke mechanism (such as shown in FIGS. 66 and 67), or other actuation assembly, examples of which are described in further detail with respect to FIGS. 62-67. Additionally or alternatively, one or more motion-transmitting components used with such actuation assemblies may include a rod, shaft, linkage, cable, wire, flexible shaft, push-pull member, or other drive member. By permitting different actuation approaches to be used with a common distal stitch-forming concept, the suturing device 100-2 may be adapted for different instrument sizes, manufacturing constraints, force-transmission requirements, access-port sizes, form factors, imagining environments, and surgical environments, including without limitation different types of surgical robots and/or different robotic actuation mechanisms.

The movable arm 338 and the magnetic coupling arrangement, described herein may be implemented in a variety of forms. For example, the movable arm 338 may include a lever, arm, link, follower, slider, carriage, or other movable transfer members. Additionally or alternatively, the magnetic coupling arrangement may include one or more magnets 314 carried by the movable arm 338 and one or more magnetically responsive components carried by the bobbin 318 and/or bobbin capsule 336, or vice versa. In further embodiments, the magnetic coupling arrangement may provide attraction, repulsion, or both, and may be configured to translate, guide, bias, retain, or otherwise influence movement of the bobbin 318 and/or bobbin capsule 336 relative to the bobbin carrier 168-2.

The configuration shown in FIG. 58 may provide one or more technical advantages. For example, the elongated and compact architecture may improve access to tissue 322 located within confined or difficult-to-reach anatomical spaces. Additionally or alternatively, the in-line arrangement of the distal components may reduce the profile of the working end 304 of the device 100-2, which may facilitate passage through a port or cannula and may improve maneuverability near target tissue. In further examples, coordinated movement of the curved needle assembly 136-2 and the bobbin 318 may improve repeatability of locking-stitch formation, may reduce reliance on complex manual needle handling within a body cavity, and may support efficient placement of sutures 142-2 in procedures where space, visibility, or instrument freedom is limited.

In various embodiments, the suturing device 100-2 and related versions described herein may be used in a wide variety of surgical procedures. By way of example and not limitation, such procedures may include closure of incisions or other openings, repair of hernias with or without mesh reinforcement, repair of diaphragmatic defects or ruptures, closure or reinforcement of gastrointestinal tissue, reinforcement of staple lines, repair of tendons, ligaments, or other soft tissues, repair of rotator cuff injuries, repair of thoracic structures, vascular repair, and veterinary procedures. In some examples, the device 100-2 may be particularly useful in repairs in which locking sutures are desirable, such as tendon repairs or other procedures benefiting from secure tissue suturing. Additionally or alternatively, the suturing device 100-2 may be useful where a surgeon desires to place a locking stitch through tissue 322 while minimizing the number of separate instrument manipulations performed within the surgical site. For example, the device 100-2 may permit a first portion of a suture 142-2 to be advanced through tissue 322 by the curved needle assembly 136-2 and a second portion of the suture 142-2 to be repositioned by bobbin movement so that the two portions of the suture 142-2 cooperate to form a locking stitch. Such a configuration may improve procedural efficiency, may reduce the difficulty associated with conventional intracorporeal suturing techniques, and may support more consistent stitch formation in minimally invasive environments.

In various embodiments, the suturing device 100-2 may be provided in one or more form factors selected according to the application. For example, in laparoscopic, thoracoscopic, and/or various robotic embodiments, the elongated body 308 may have a length in a range of approximately 30 cm to approximately 34 cm. In another example, for arthroscopic configurations, the elongated body 308 may be shorter than laparoscopic and/or thoracoscopic configurations to accommodate the shorter access distance associated with joint procedures. In various embodiments, the suturing device 100-2 may be configured for introduction through a trocar, cannula, or other access port, and, thus, the outer dimension of the elongated body 308 may be selected according to the access-port size. For example, embodiments configured for use with a nominal 5 mm port may have an outer diameter of less than 5 mm, such as approximately 4 mm. In another example, embodiments configured for use with a nominal 12 mm port may have an outer diameter of less than 12 mm, such as approximately 11 mm. It will be appreciated that other lengths, diameters, and form factors may be used depending on anatomy, access path, robotic interface, procedure type, and desired mechanical strength.

FIG. 60 illustrates a side view of a distal portion 304 of the suturing device 100-2 with a curved needle assembly 136-2 in a retracted position 350, and FIG. 61 illustrates a side view of the distal portion 304 of the suturing device 100-2 of FIG. 60 with the curved needle assembly 136-2 in an extended position 352, in accordance with embodiments of the invention. As previously discussed, in various embodiments, the distal portion 304 of the elongated body 308 may include the curved needle assembly 136-2, the bobbin carrier 168-2, the bobbin 318, and/or the bobbin capsule 336 arranged in a compact stitch-forming configuration. In various embodiments, the curved needle assembly 136-2 may be movable relative to the elongated body 308 between the retracted position 350 and the extended position 352. For example, the curved needle assembly 136-2 may be retracted within, adjacent to, or otherwise positioned relative to the distal portion 304 such that the curved needle assembly 136-2 is prepared for placement adjacent to tissue 322 before advancement of the suture 142-2. In various embodiments, the curved needle assembly 136-2 of the suturing device 100-2 may operate in a manner similar to the curved suturing needle assembly 136 of the suturing device 100 described above. For example, the curved needle assembly 136-2 may be actuated to advance a first portion of the suture 142-2 along a curved suture path and at least part way through tissue 322 as the curved needle assembly 136-2 moves toward the extended position 352. In various embodiments, such movement toward the extended position 352 may form a bow with the first portion of the suture 142-2. The bobbin 318 may then be moved relative to the bobbin carrier 168-2 so that a second portion of the suture 142-2 passes through the bow. Thereafter, the curved needle assembly 136-2 may be moved toward the retracted position 350 to retract the first portion of the suture 142-2, thereby forming a locking stitch and securing the suture at the tissue 322.

In various embodiments, when the curved needle assembly 136-2 is in the retracted position 350, as shown in FIG. 60, the curved needle assembly 136-2 may be positioned so as to reduce the profile of the distal portion 304 to facilitate placement of the suturing device 100-2 relative to the tissue 322. The retracted position 350 may correspond to a pre-actuation state in which a first portion of the suture 142-2 is positioned for subsequent advancement through the tissue 322.

Actuation of the control assembly 300 may cause the curved needle assembly 136-2 to move from the retracted position 350 (e.g., FIG. 60) toward the extended position 352 (e.g., FIG. 61). In various embodiments, the curved needle 380 of the curved needle assembly 136-2 may be operably coupled with a pivoting arm 128-2 supported relative to the elongated body 308 at a fulcrum 134-2. Additionally, movement transmitted from the control assembly 300 may be delivered through a needle driving axle 342, which may be coupled with the pivoting arm 128-2 by one or more movable joints 129-2. In this way, motion originating at the control assembly 300 may be translated to the curved needle assembly 136-2 to cause the curved needle assembly 136-2 to rotate so as to advance the suture 142-2 along the suture path through the tissue 322. For example, as shown in FIG. 61, movement of the curved needle assembly 136-2 to the extended position 352 may advance a first portion of the suture 142-2 along a suture path through the tissue 322. In various embodiments, the curved needle assembly 136-2 may pass through one or more slots 328 of the bobbin carrier 168-2 during such movement. The one or more slots 328 may thereby provide clearance for movement of the curved needle assembly 136-2 while permitting the bobbin carrier 168-2, bobbin 318, and/or bobbin capsule 336 to remain in a compact arrangement at the distal portion 304.

In various embodiments, the extended position 352 may correspond to a stitch-forming position in which a first portion of the suture 142-2 has been advanced through the tissue 322 and is positioned for cooperation with a second portion of the suture 142-2 carried by the bobbin 318. In various embodiments, the curved needle assembly 136-2 may advance through the tissue 322 while the bobbin carrier 168-2 remains fixed relative to the elongated body 308. Such a configuration may permit the curved needle assembly 136-2 and the bobbin-related components to operate in close proximity while preserving the relatively narrow profile of the distal portion 304.

In various embodiments, the bobbin 318 may be retained within a bobbin capsule 336, which may support the bobbin 318 for movement relative to the bobbin carrier 168-2. In various embodiments, a portion of the bobbin 318 and/or bobbin capsule 336 may include a magnetically responsive component, shown in FIGS. 60 and 61 as a belt 340 positioned on or around the bobbin capsule 336. The magnetically responsive component (e.g., belt 340) may be magnetically couplable with a magnet 314 carried by the movable arm 338 (illustrated in FIG. 59), such as the magnet 314 discussed above with respect to FIG. 59. In various embodiments, the magnetically responsive component may include a belt 340, band, ring, collar, sleeve, insert, plate, coating, embedded member, or other structure positioned on, within, around, or coupled to the bobbin 318 and/or bobbin capsule 336. In various embodiments, the magnet 314 may include a permanent magnet, electromagnet, magnet array, or other magnetic element. Movement of the movable arm 338 may cause corresponding movement of the bobbin capsule 336 and/or bobbin 318 through magnetic coupling between the magnet 314 of the movable arm 338 and the magnetically responsive component or belt 340. Such a configuration may permit bobbin movement to be produced without requiring a bulky internal mechanical drive member to pass directly through the bobbin carrier 168-2, thereby supporting a compact distal architecture. In various embodiments, the bobbin carrier 168-2 may include a groove 332 configured to guide movement of the movable arm 338 relative to the bobbin carrier 168-2. For example, a portion of the movable arm 338 including the magnet 314 may extend into the groove 332 such that the groove 332 constrains the travel of the movable arm 338 along a desired path, which may improve alignment between the magnet 314 and the magnetically responsive component (e.g., belt 340) of the bobbin capsule 336 during actuation. In this way, the groove 332 may facilitate controlled movement of the bobbin capsule 336 and/or bobbin 318 relative to the bobbin carrier 168-2.

After the curved needle assembly 136-2 reaches the extended position 352, the bobbin 318 may be moved relative to the bobbin carrier 168-2 so that the second portion of the suture 142-2 is positioned relative to the first portion of the suture 142-2 advanced by the curved needle assembly 136-2. In various embodiments, such bobbin movement may occur while the curved needle assembly 136-2 is in the extended position 352, while the curved needle assembly 136-2 is transitioning to the extended position 352, or while the curved needle assembly 136-2 is in the retracted position 350. Thereafter, the curved needle assembly 136-2 may be returned toward the retracted position 350, thereby retracting the first portion of the suture 142-2 and facilitating formation of a locking stitch in the tissue 322.

In various embodiments, the positions shown in FIGS. 60 and 61 may illustrate one example of a needle actuation sequence that may be used in the suturing device 100-2. For example, the retracted position 350 may provide a low-profile starting configuration for placement and alignment of the distal portion 304, while the extended position 352 may permit controlled advancement of the suture 142-2 through the tissue 322. This arrangement may improve the ability of the suturing device 100-2 to place sutures within confined anatomical spaces and may support repeatable formation of locking stitches in minimally invasive procedures.

The compact interaction between the curved needle assembly 136-2, the bobbin carrier 168-2, the bobbin 318, and/or bobbin capsule 336 shown in FIGS. 60 and 61 may provide one or more technical advantages. For example, the use of the one or more slots 328 may permit the curved needle assembly 136-2 to travel through the distal portion 304 without requiring a larger lateral footprint. Additionally or alternatively, maintaining the bobbin carrier 168-2 in a fixed position while allowing the curved needle assembly 136-2 to move between the retracted position 350 and the extended position 352 may assist in preserving a narrow distal profile suitable for laparoscopic, arthroscopic, and robot-assisted procedures. In further examples, magnetic coupling between the movable arm 338 and a magnetically responsive component (e.g., belt 340) of the bobbin 318 and/or bobbin capsule 336 may facilitate controlled movement of the bobbin-related components while reducing packaging constraints at the distal portion 304.

In various embodiments, one or more actuated components, such as the handle lever 108, handle lever 108-2, thumb lever 200, thumb lever 212, and/or thumb lever 310, may be spring loaded and/or otherwise biased. In various embodiments, the control assembly 300 may be configured such that actuation occurs when a predetermined range of force is applied. For example, actuation of one or more portions of the control assembly 300, such as the handle lever 108-2 or the thumb lever 310, may be configured to occur when a force of 20 N to 50 N is applied such that incidental bumping, unintended contact, or minor disturbances are less likely to cause unintentional deployment of the curved needle assembly 136-2 and/or movement of the bobbin 318. Accordingly, the actuation force, spring rate, trigger travel, latch geometry, trigger geometry, and/or mechanical advantage of the actuation system may be selected to balance ease of use with resistance to inadvertent actuation.

FIG. 62 illustrates a side view of the distal portion 304 of the suturing device 100-2 including a bell crank actuation assembly 324, and FIG. 63 illustrates a side view of the distal portion 304 of the suturing device 100-2 including another bell crank actuation assembly 324, in accordance with embodiments of the invention. In various embodiments, the bell crank actuation assemblies 324 shown in FIGS. 62 and 63 may be configured to transmit and/or redirect motion within the distal portion 304 of the suturing device 100-2. For example, the bell crank actuation assembly 324 may be operably coupled with one or more motion-transmitting components extending through the elongated body 308, such as component 348, and may further be operably coupled with the movable arm 338 configured to move the bobbin 318 and/or the bobbin capsule 336 relative to the bobbin carrier 168-2. In this way, motion delivered from the control assembly 300 may be translated by the bell crank actuation assembly 324 into movement of the movable arm 338 at the distal portion 304. In various embodiments, the bell crank actuation assemblies 324 shown in FIGS. 62 and 63 may be implemented in minimally invasive embodiments, such as laparoscopic, arthroscopic, thoracoscopic, endoscopic, and robot-assisted versions, and may additionally or alternatively be implemented in larger-format, open, or handheld embodiments, such as the suturing device 100 described above.

In various embodiments, the bell crank actuation assembly 324 may include a pivotable member supported relative to the elongated body 308 or another support structure (e.g., component 348) of the suturing device 100-2. The pivotable member may include a first portion 358 configured to receive motion from the component 348, a second portion 372 configured to transmit corresponding motion to the movable arm 338, and a pivot 374 coupling the first portion 358 with the second portion 372. In various embodiments, a spring 330 may further be coupled to the pivotable member. For example, a spring 330 may be operably associated with the second portion 372 as illustrated in FIGS. 62 and 63. In various embodiments, the second portion 372 may be actuated and subsequently released to produce movement of the movable arm 338. For example, the second portion 372 may be actuated by the bell crank lever (e.g., first portion 358) as the handle lever 108-2 is released against spring force provided by spring 330. Thereafter, subsequent actuation of the handle lever 108-2, such as during squeezing of the handle lever 108-2 by a user, may permit release of energy stored in the spring 330 through movement of the second portion 372. In this way, motion input at the proximal end 306 may be used to store energy in the spring 330 and then release that energy through the second portion 372 to drive movement of the movable arm 338. As a result, the substantially longitudinal movement of the component 348 may be converted by the bell crank actuation assembly 324 into transverse, angular, curvilinear, and/or otherwise redirected movement of the movable arm 338. Such movement of the movable arm 338 may, in turn, cause the bobbin 318 to move relative to the bobbin carrier 168-2, such as via the magnetic configuration discussed above which employs a magnet 314 carried by the movable arm 338 magnetically coupled with the belt 340 of the bobbin 318 and/or bobbin capsule 336. In various embodiments, the bell crank actuation assemblies 324 shown in FIGS. 62 and 63 may include one or more stop rings, stops, collars, shoulders, or other limiting structures coupled with component 348. The one or more limiting structures may limit movement of component 348 and thereby define a predetermined range of movement for the bell crank actuation assembly 324.

In various embodiments, the bell crank actuation assemblies 324 shown in FIGS. 62 and 63 may further include a trigger mechanism 384 configured to selectively retain and release stored energy, such as spring energy, used to drive movement of the movable arm 338. For example, one or more pivots 374 may be associated with the trigger mechanism 384 and may be spring loaded, such as by a coil spring 330, so that the trigger mechanism 384 is biased toward a retaining position. Then actuation of the handle lever 108-2 may cause the trigger mechanism 384 to release the stored spring energy, thereby permitting movement of the second portion 372 and driving the movable arm 338.

In various embodiments, the bell crank actuation assembly 324 shown in FIG. 62 may provide one example in which motion delivered to the distal portion 304 may be redirected to move the movable arm 338 along a desired path relative to the bobbin carrier 168-2. For example, the bell crank actuation assembly 324 may convert input motion from the component 348 into movement of the movable arm 338 along the groove 332 of the bobbin carrier 168-2. In such embodiments, travel of the movable arm 338 along the groove 332 may maintain alignment between the magnet 314 and the magnetically responsive component of the bobbin capsule 336 during bobbin actuation. This may facilitate controlled translation of the bobbin 318 relative to the bobbin carrier 168-2 for positioning a second portion of the suture 142-2 relative to the first portion of the suture 142-2 advanced by the curved needle assembly 136-2.

In various embodiments, the bell crank actuation assembly 324 shown in FIG. 63 may provide another example of a bell crank-based actuation architecture. For example, the FIG. 63 arrangement may include a trigger mechanism 384 having a portion movable about one or more pivots 374. A cone-shaped or tapered portion 382 of the trigger mechanism 384 may pass into or through a trigger guard 386 and may contact a portion of the trigger mechanism 384, thereby moving the trigger mechanism 384 aside, such as laterally and/or rotationally about one or more pivots 374, to release the spring 330. Release of the spring 330 may then permit spring-driven movement of the second portion 372 and corresponding movement of the movable arm 338. In various embodiments, such differences may alter stroke length, force transmission, timing, mechanical advantage, packaging, and/or the path of movement imparted to the movable arm 338. Accordingly, FIG. 63 may illustrate that a variety of bell crank configurations may be used to move the bobbin 318 and/or bobbin capsule 336 relative to the bobbin carrier 168-2 while maintaining the general locking-stitch functionality described herein.

FIG. 64 illustrates a side view of the distal portion 304 of the suturing device 100-2 including a linear actuator assembly 354, and FIG. 65 illustrates a side view of the distal portion 304 of the suturing device 100-2 including another linear actuator assembly 354, in accordance with embodiments of the invention. In various embodiments, the linear actuator assemblies 354 shown in FIGS. 64 and 65 may be configured to transmit motion within the distal portion 304 of the suturing device 100-2 for purposes of moving the bobbin 318 and/or the bobbin capsule 336 relative to the bobbin carrier 168-2 and/or moving the curved needle assembly 136-2. For example, the linear actuator assembly 354 may be operably coupled with one or more motion-transmitting components extending through the elongated body 308, such as component 348, and may further be operably coupled with the movable arm 338. In this way, motion delivered from the control assembly 300 may be translated by the linear actuator assembly 354 into substantially linear movement at the distal portion 304. In various embodiments, the linear actuator assemblies 354 shown in FIGS. 64 and 65 may be implemented in minimally invasive embodiments, such as laparoscopic, arthroscopic, thoracoscopic, and robot-assisted versions, and may additionally or alternatively be implemented in larger-format, open, or handheld embodiments, such as the suturing device 100 described above.

In various embodiments, the linear actuator assembly 354 shown in FIG. 64 may include a ball screw arrangement 364 configured to convert substantially linear input motion into rotational output motion. For example, linear motion transmitted from the control assembly 300, such as by component 348, may be delivered to the linear actuator assembly 354, and the linear actuator assembly 354 may translate that linear motion into rotation of the movable arm 338. In various embodiments, the ball screw arrangement 364 may include one or more rolling elements (e.g., components 376), such as ball bearings or other bearing members, positioned to reduce friction and facilitate repeatable motion transfer. In various embodiments, the ball screw arrangement 364 may include a threaded member 378 and a cooperating component (e.g., components 376) configured to translate linear motion from the control assembly 300 to rotational motion of the movable arm 338.

In various embodiments, the linear actuator assembly 354 shown in FIG. 65 may provide another example of a linear actuator-based architecture. For example, the arrangement shown in FIG. 65 may differ from the arrangement shown in FIG. 64 with respect to the relative positioning of the threaded member 378 and/or the cooperating component 376, and may further differ in stroke path, output orientation, packaging, and/or force transmission characteristics.. In various embodiments, such differences may alter the path of movement imparted to the movable arm 338, the magnitude of bobbin displacement, the force transmitted to the bobbin 318 and/or bobbin capsule 336, and/or other operating characteristics. Accordingly, FIG. 65 may illustrate that a variety of linear actuator configurations may be used while maintaining the general locking-stitch functionality described herein.

FIG. 66 illustrates a side view of the distal portion 304 of the suturing device 100-2 including a scotch yoke actuator assembly 356, and FIG. 67 illustrates a side view of the distal portion 304 of the suturing device 100-2 including another scotch yoke actuator assembly 356, in accordance with embodiments of the invention. In various embodiments, the scotch yoke actuator assemblies 356 shown in FIGS. 66 and 67 may be configured to transmit motion within the distal portion 304 of the suturing device 100-2 for purposes of moving the bobbin 318 and/or the bobbin capsule 336 relative to the bobbin carrier 168-2. For example, the scotch yoke actuator assembly 356 may be operably coupled with one or more motion-transmitting components extending through the elongated body 308, such as component 348, and may further be operably coupled with the movable arm 338. In this way, motion delivered from the control assembly 300 may be translated by the scotch yoke actuator assembly 356 into movement of the movable arm 338 at the distal portion 304.

In various embodiments, the scotch yoke actuator assembly 356 shown in FIG. 66 may include a first driven member 370a, a second driven member 370b, and a slotted component 368. The slotted component 368 may be configured to translate the generally linear motion of component 348 from the control assembly 300 to rotational motion of the first driven member 370a. In turn the rotational motion of the first driven member 370a may transfer to the second driven member 370b to be converted into an output motion of the movable arm 338. In various embodiments, the first and second driven members 370 may be one or more gears, as illustrated, or may be any other components suitable to transfer motion. In various embodiments, the arrangement shown in FIG. 66 may be particularly suitable for minimally invasive configurations, such as laparoscopic embodiments, where distal components may be arranged generally in-line with the longitudinal axis 312, thereby minimizing the overall size of the device 100-2. Additionally or alternatively, the arrangement of FIG. 66 may be adapted for use in larger-format, open, or handheld embodiments, including embodiments similar to suturing device 100.

In various embodiments, the scotch yoke actuator assembly 356 shown in FIG. 66 may provide one example in which motion is converted in a relatively compact and repeatable manner to move the movable arm 338 along a desired path relative to the bobbin carrier 168-2. For example, the scotch yoke actuator assembly 356 may be utilized with a bobbin carrier 168-2 that is generally aligned with the longitudinal axis 312. As a result, the overall size of the suturing device 100-2 may be reduced. The scotch yoke actuator assembly 356 shown in FIG. 67 may provide one example in which motion is converted when the bobbin carrier 168-2 is not generally aligned with the longitudinal axis 312, such as with the first embodiment of the handheld suturing device 100 discussed herein. For example, the bobbin carrier 168-2 in such embodiments need not be generally aligned with the longitudinal axis 312. Accordingly, FIG. 67 may illustrate that a variety of scotch yoke configurations may be used while maintaining the general locking-stitch functionality described herein. In various embodiments, the arrangement shown in FIG. 67 may be particularly suitable for open, handheld, or other larger-format implementations, such as suturing device 100.

In various embodiments, the actuation assemblies described with respect to FIGS. 62-67, including the bell crank actuation assemblies 324, the linear actuator assemblies 354, and the scotch yoke actuator assemblies 356, may be coordinated with actuation of both the curved needle assembly 136-2 and the bobbin 318. For example, the motion-transmitting component 342 associated with the curved needle assembly 136-2 and the motion-transmitting component 348 associated with movement of the bobbin 318 and/or bobbin capsule 336 may both be activated by the handle lever 108-2. As a result, movement of the handle lever 108-2 may control both needle movement and bobbin actuation, optionally in a coordinated or substantially simultaneous manner. In various embodiments, movement of the curved needle assembly 136-2 and movement of the bobbin 318 and/or bobbin capsule 336 may occur simultaneously, overlap in time, or occur in partially overlapping motion sequences. Additionally or alternatively, such movements may occur sequentially. In various embodiments, the actuation assemblies described herein may additionally or alternatively be used with a control arrangement employing both the handle lever 108-2 and the thumb lever 310, or with a control arrangement employing a single lever configured to trigger multiple device motions. For example, the handle lever 108-2 may actuate the curved needle assembly 136-2 and the thumb lever 310 may cause movement of the bobbin 318. Alternatively, a single universal lever may be configured to trigger all or substantially all of the relevant movements of the suturing device 100-2, including movement of the curved needle assembly 136-2 and movement of the bobbin 318 and/or bobbin capsule 336 relative to the bobbin carrier 168-2.

The actuation assemblies described with respect to FIGS. 62-67 may provide one or more technical advantages. For example, the bell crank, linear actuator, and scotch yoke variations may permit different forms of motion delivered generally along the longitudinal axis 312 of the elongated body 308 to be converted into controlled movement suitable for repositioning the bobbin 318 and/or bobbin capsule 336 relative to the bobbin carrier 168-2. Additionally, such variations may provide flexibility in actuator geometry, force transmission, stroke length, timing, packaging, mechanical advantage, manufacturability, and ergonomic configuration while maintaining a compact distal architecture.

In various embodiments, the configurations, mechanisms, and methods described with respect to suturing device 100-2 are not limited to minimally invasive formats, such as laparoscopic, arthroscopic, thoracoscopic, endoscopic, and robot-assisted procedures, and may additionally or alternatively be implemented in various form factors and configurations, such as in connection with open surgery like suturing device 100. For example, the suturing device 100-2 may be scaled, enlarged, or otherwise reconfigured for open surgical use and/or manual manipulation by a user, while maintaining corresponding functional relationships among the components so that the device 100-2 may be used in a manner similar to the handheld medical suturing device 100 described above. In this way, the embodiments described with respect to suturing device 100-2 may be adapted to different scales, access modalities, and use environments, including minimally invasive, open, manual, powered, and robotic implementations.

In various embodiments, any of the suturing devices described herein, including the handheld medical suturing device 100 and the suturing device 100-2, may be configured for single-use and disposal after a single procedure and/or after use with a single patient. Alternatively, any of the suturing devices described (e.g., suturing device 100 and suturing device 100-2) may include one or more components intended for a predetermined number of use cycles before replacement. Additionally or alternatively, portions of any of the suturing devices described (e.g., suturing device 100 and suturing device 100-2) may be configured to be reusable while other portions may be configured as disposable, such as where one or more distal portions are tissue-penetrating portions are configured for disposal and one or more proximal actuation portions, robotic interface portions, or non-patient-contact portions are configured for reuse.

In various embodiments, the devices described herein (e.g., suturing device 100 and suturing device 100-2) may be formed from one or more metals, polymers, composites, ceramics, and/or combinations thereof. For example, the elongated body 308 and at least a portion of the control assembly 300 may be formed from one or more rigid polymeric materials, while the curved needle assembly 136-2 may be formed from one or more metallic materials. Suitable metallic materials may include, without limitation, stainless steel, surgical steel, non-ferromagnetic stainless steel, aluminum, titanium, and/or alloys thereof.

In various embodiments, the devices described herein (e.g., suturing device 100 and suturing device 100-2) may be utilized with one or more imaging modalities during placement, positioning, actuation, or suturing. For example, the suturing device 100-2 may be utilized along with various guidance forms such as visual, ultrasound, fluoroscopic, computed tomography (CT or CAT), magnetic resonance imaging (MRI), and/or combinations thereof. In various embodiments, one or more components may be formed from or include non-ferromagnetic materials to facilitate use in environments in which magnetic compatibility is desired. For example, where use in an MRI environment is desired, some or all device components may be formed of non-ferromagnetic materials to reduce magnetic interaction.

In various embodiments, one or more portions of the devices described herein (e.g., suturing device 100 and suturing device 100-2) and/or sutures may include one or more surface treatments, coatings, impregnations, or medicaments. For example, one or more sutures may be antibiotic-impregnated and/or may include one or more antimicrobial agents. In another embodiments, one or more portions of the suture device 100-2 may include lubricious coatings, low-friction coatings, anti-stick coatings, hydrophobic coatings, antimicrobial coatings, antiseptic coatings, corrosion-resistant coatings, wear-resistant coatings, and/or combinations thereof.

While particular embodiments and applications have been illustrated and described herein, it is to be understood that the embodiments are not limited to the precise construction and components disclosed herein and that various modifications, changes, and variations may be made in the arrangement, operation, and details of the methods and apparatuses of the embodiments without departing from the spirit and scope of the embodiments as defined in the appended claims.

Upon reading this disclosure, those of skill in the art will appreciate still additional alternative designs for the system. Thus, while particular embodiments and applications of the present disclosure have been illustrated and described, it is to be understood that the disclosure is not limited to the precise construction and components disclosed herein and that various modifications, changes and variations which will be apparent to those skilled in the art may be made in the arrangement, operation and details of the method and apparatus of the present disclosure disclosed herein without departing from the spirit and scope of the disclosure as defined in any claims drawn to the subject matter herein.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

This invention is susceptible to considerable variation within the spirit and scope of the appended claims.

All U.S. patents and all publications identified herein are incorporated in their entirety by reference thereto.

What is claimed is:

1. A suturing device comprising:
an elongated body having a proximal end opposite a distal end, the elongated body defining a longitudinal axis;
a curved needle assembly arranged at the distal end of the elongated body and movable between at least an extended position and a retracted position, the curved needle assembly configured, in the extended position, to advance a first portion of a suture along a suture path through tissue, and configured, in the retracted position, to retract the first portion of the suture along the suture path; and
a control assembly arranged at the proximal end of the elongated body, the control assembly configured to move the curved needle assembly.

2. The suturing device of claim 1, further comprising:
a bobbin carrier arranged at the distal end of the elongated body, the bobbin carrier aligned with the curved needle assembly along the longitudinal axis;
a bobbin positioned within the bobbin carrier and configured to carry a second portion of the suture; and
a movable arm operably coupled to the bobbin and configured to move the bobbin relative to the bobbin carrier.

3. The suturing device of claim 2, wherein the control assembly is configured to move at least one of the curved needle assembly and the bobbin so as to form a locking stitch in the tissue between the first portion of the suture and the second portion of the suture.

4. The suturing device of claim 3, wherein the curved needle assembly moving to the extended position further includes forming a bow with the first portion of the suture, wherein the locking stitch is formed by moving the second portion of the suture via the bobbin through the bow and moving the curved needle assembly to the retracted position.

5. The suturing device of claim 1, wherein, in the extended position, a curved needle of the curved needle assembly is advanced part way through the tissue.

6. The suturing device of claim 2, wherein the bobbin carrier defines an opening at a distal end, the opening configured to receive the tissue.

7. The suturing device of claim 2, wherein the control assembly includes at least a first actuator configured to move the curved needle assembly, and a second actuator configured to move the bobbin via the movable arm.

8. The suturing device of claim 2, wherein the bobbin carrier defines a groove and at least a portion of the movable arm is configured to travel along the groove to move the bobbin relative to the bobbin carrier.

9. The suturing device of claim 8, further comprising a magnet arranged on the movable arm, wherein the magnet is magnetically coupled with at least a portion of the bobbin such that movement of the movable arm causes corresponding movement of the bobbin relative to the bobbin carrier.

10. The suturing device of claim 9, wherein the portion of the bobbin magnetically coupled to the magnet is a ferromagnetic ring arranged around at least a portion of the bobbin.

11. The suturing device of claim 2, further comprising a scotch yoke mechanism operably coupled to the movable arm, the scotch yoke mechanism comprising a yoke and a gear set configured to convert movement generated by actuation of the control assembly into movement of the movable arm.

12. The suturing device of claim 2, wherein the bobbin carrier is fixed relative to the elongated body, and the bobbin is movable within the bobbin carrier.

13. The suturing device of claim 12, further comprising a bobbin capsule positioned within the bobbin carrier, the bobbin capsule configured to retain the bobbin therein.

14. The suturing device of claim 2, wherein the curved needle assembly includes an arm coupled with the bobbin carrier at a fulcrum point, wherein movement of the curved needle assembly between the retracted position and the extended position is defined by pivoting of the arm about the fulcrum point.

15. The suturing device of claim 1, wherein the control assembly comprises a robot.

16. The suturing device of claim 1, wherein the suturing device is adapted for use with a laparoscope or arthroscope.

17. A method of forming a locking stitch in tissue with a suturing device, the method comprising:
positioning a curved needle assembly adjacent to tissue, the curved needle assembly arranged at a distal end of an elongated body;
actuating a control assembly arranged to cause the curved needle assembly to move to an extended position and to advance a first portion of a suture along a suture path through the tissue, the control assembly arranged at a proximal end of the elongated body;
actuating the control assembly to cause the curved needle assembly to move to a retracted position, thereby retracting the first portion of the suture along the suture path and securing the suture at the tissue.

18. The method of claim 17, further comprising:
actuating the control assembly to move a bobbin relative to a bobbin carrier arranged at the distal end of the elongated body, wherein moving the curved needle assembly to the extended position further includes forming a bow with the first portion of the suture, wherein movement of the bobbin causes a second portion of the suture carried by the bobbin to move through the bow; and
actuating the control assembly to cause the curved needle assembly to move to a retracted position, thereby retracting the first portion of the suture along the suture path and forming a locking stitch at the tissue with the second portion of the suture.

19. The method of claim 17, wherein, in the extended position, a curved needle of the curved needle assembly is advanced part way through the tissue.

20. The method of claim 17, further comprising positioning at least a portion of the tissue within an opening defined at a distal end of the bobbin carrier.

21. The method of claim 17, wherein actuating the control assembly to cause the curved needle assembly to move to the extended position comprises actuating a first actuator of the control assembly.

22. The method of claim 21, wherein actuating the control assembly to move the bobbin relative to the bobbin carrier comprises actuating a second actuator of the control assembly.

23. The method of claim 17, wherein moving the bobbin relative to the bobbin carrier comprises moving at least a portion of a movable arm along a groove defined by the bobbin carrier, wherein the movable arm is operably coupled with the bobbin.

24. The method of claim 23, wherein moving the bobbin relative to the bobbin carrier further comprises magnetically coupling a magnet on the movable arm with at least a portion of the bobbin, such that movement of the movable arm causes corresponding movement of the bobbin.

25. The method of claim 24, wherein the movable arm is arranged outside a wall of the bobbin carrier such that the bobbin is moved through the wall of the bobbin carrier by the magnetic coupling.

26. The method of claim 17, wherein the bobbin carrier remains fixed relative to the elongated body while the bobbin is moved within the bobbin carrier.

27. The method of claim 17, wherein moving the bobbin relative to the bobbin carrier comprises actuating at least one of a scotch yoke mechanism operably coupled to the movable arm or a ball screw actuator operably coupled to the movable arm.

28. The method of claim 17, wherein the actuation of the control assembly comprises a robot.

29. The method of claim 17, further comprising forming a plurality of locking stitches in tissue in one or more of the following: repair of hernias with or without mesh reinforcement, repair of diaphragmatic defects or ruptures, closure or reinforcement of gastrointestinal tissue, reinforcement of staple lines, repair of tendons, ligaments, or other soft tissues, repair of rotator cuff injuries, repair of thoracic structures, and vascular repair.

30. The method of claim 17, wherein the tissue is human tissue.

31. The method of claim 17, wherein the tissue is animal tissue.
